(19) United States
(12) Patent Application Publication
(10) Pub. No.: US 2026/0318825 A1
(43) Pub. Date: Oct. 15, 2026

(54) MULTI-LASER SPECTROSCOPY SYSTEMS AND RELATED METHODS

(71) Applicant: Gemological Institute of America, Inc. (GIA), Carlsbad, CA (US)

(72) Inventors: Simon Lawson, Berkshire (GB); Kevin De Leon, Carlsbad, CA (US); Mehdi Toosi, Carlsbad, CA (US); Rj Pisani, Carlsbad, CA (US); Penghui Yang, Carlsbad, CA (US); Troy Ardon, Carlsbad, CA (US); Daniel Jones, Carlsbad, CA (US)

(21) Appl. No.: 19/646,393

(22) Filed: Apr. 13, 2026

Related U.S. Application Data
(60) Provisional application No. 63/789,318, filed on Apr. 15, 2025.

(51) Int. Cl. G01N 21/31; G01N 1/42; G01N 33/00

(52) U.S. Cl. CPC G01N 21/31; G01N 1/42; G01N 33/389; G01N 2201/06113; G01N 2201/08

(57) ABSTRACT

Multi-laser spectroscopy systems and related methods are disclosed. The multi-laser spectroscopy systems as described herein can include a laser device with multiple laser sources and an optical combiner to combine laser light from the multiple laser sources to generate incident light. The incident light can be directed to a sample disposed in a chamber to interact with the sample, thereby generating responsive light directed to a spectrometer configured to generate measurement signals corresponding to the responsive light. A computing device can cause laser sources to excite laser light in different combinations to alter the incident light and obtain the measurement signals generated by the spectrometer to determine one or more characteristics of the sample.

### CROSS-REFERENCE TO RELATED APPLICATIONS

The present application claims priority of U.S. Provisional Application No. 63/789,318, titled “MULTI-LASER SPECTROSCOPY SYSTEMS AND RELATED METHODS,” and filed on Apr. 15, 2025, the entirety of which is incorporated by reference herein.

### TECHNICAL FIELD

This disclosure relates generally to multi-laser spectroscopy systems and related methods, and more specifically to multi-laser spectroscopy systems and methods for making automated multi-laser high-spectral-resolution photoluminescence (PL) and Raman measurements and analysis of samples (e.g., gemstones) at liquid nitrogen temperatures.

### BACKGROUND

Photoluminescence (PL) and Raman spectroscopy are non-destructive analytical techniques for characterizing aspects of a gemstone, such as determining diamond types or whether the gemstone is of a natural or a synthetic origin. The process can generally include exposing a sample to a light source, and recording the spectrum emitted from the sample. The light source can include any of a laser, a light-emitting diode (LED), or a flash lamp, for example. In these systems, light of different wavelengths are often used for excitation, because light can only excite emission with an equivalent or lower amount of energy. For example, with a diamond, lasers with different wavelengths can be used to excite multiple targeted defect centers. In this example, to have high-resolution spectra, a liquid nitrogen temperature or equivalent can be needed during the PL measurement of a diamond, because the emission feature can be sharper, having narrower full width at half maximum (FWMH), at liquid nitrogen temperature.

Such PL and Raman systems with multiple laser sources and a low temperature measurement capability may be expensive, large in space, time consuming to use and can often require a high level of skill for operation. It is desirable to have a instrument that can be a smaller desktop unit that is cheaper and easy to use but still has similar functionality such as comprising multiple excitation wavelengths and liquid nitrogen temperature measurement capabilities.

### SUMMARY

Multi-laser spectroscopy systems and related methods are disclosed. The multi-laser spectroscopy systems as described herein can include a laser device with multiple laser sources and an optical combiner to combine laser light from the multiple laser sources to generate incident light. The incident light can be directed to a sample disposed in a chamber to interact with the sample, thereby generating responsive light directed to a spectrometer configured to generate measurement signals corresponding to the responsive light. A computing device can cause laser sources to excite laser light in different combinations to alter the incident light and obtain the measurement signals generated by the spectrometer to determine one or more characteristics of the sample.

In a first example embodiment, a multi-laser spectroscopy system is provided. The multi-laser spectroscopy system can include a laser device that includes multiple laser sources configured to excite a laser light, and an optical combiner disposed adjacent to the multiple laser sources. The optical combiner can be configured to combine the excited laser light from the multiple laser sources and output incident light.

The multi-laser spectroscopy system can also include a set of laser switches electrically connected to any of the multiple laser sources. The multi-laser spectroscopy system can also include a cryochamber disposed adjacent to the laser device. The cryochamber can be configured to receive a sample and the incident light output from the optical combiner. The incident light can be configured to interact with the sample, thereby producing responsive light.

The multi-laser spectroscopy system can also include a spectrometer disposed adjacent to the cryochamber and configured to receive the responsive light and generate one or more measurement signals corresponding to the responsive light. The multi-laser spectroscopy system can also include a computing device electrically connected to the spectrometer and the set of laser switches. The computing device can be configured to cause the set of laser switches to individually control the excitation of the laser light from any of the multiple laser sources and to receive one or more measurement signals from the spectrometer to determine one or more characteristics of the sample.

In some instances, the computing device is further configured to initiate a sequence that causes the set of laser switches to individually control the excitation of the laser light from any of the multiple laser sources and thereby alter the incident light provided to the sample over a time period, and wherein the one or more measurement signals received at the spectrometer are configured to change during the time period.

In some instances, the multi-laser spectroscopy system can also include a bifurcated fiber including a probe tip to deliver the incident light to the cryochamber and receive the responsive light from the sample in response to the incident light to provide the responsive light to the spectrometer.

In some instances, the multiple laser sources include a first laser source to produce first laser light having a center frequency corresponding to a wavelength of around 457 nm, a second laser source to produce second laser light having a center frequency corresponding to a wavelength of around 515 nm, a third laser source to produce third laser light having a center frequency corresponding to a wavelength of around 633 nm, and a fourth laser source to produce fourth laser light having a center frequency corresponding to a wavelength of around 830 nm.

In some instances, the multiple laser sources include at least four laser sources.

In some instances, the optical combiner includes a network of dichroic beam splitters.

In some instances, the cryochamber includes a cryochamber housing defining the cryochamber, the probe tip extending through the cryochamber housing. The cryochamber can also include rails extending from the cryochamber housing. The cryochamber can also include a bracket including slots to receive the rails and a holder passage aligned with the probe tip responsive to the rails being received into the slots. The cryochamber can also include a sample holder extending from the holder passage of the bracket toward the probe tip, the sample holder to secure the sample at its tip, the sample holder to place the sample in contact with the probe tip.

In some instances, the bracket is shaped to define space between the bracket and the cryochamber with the sample deployed in contact with the probe tip.

In some instances, the sample holder includes holder arms extending from a tip of the sample holder, the holder arms to secure the sample to the tip of the sample holder.

In some instances, the sample holder includes an adhesive at a tip of the sample holder, the adhesive to secure the sample to the tip of the sample holder.

In some instances, the sample includes a gemstone and the sample holder secures the gemstone with a table of the gemstone facing outward away from the sample holder.

In some instances, the probe tip includes a tip of an excitation fiber surrounded by tips of detection fibers arranged in an annular pattern around the tip of the excitation fiber, the excitation fiber to extend from the optical combiner to the cryochamber, the detection fibers to extend from the cryochamber to the spectrometer.

In some instances, tips of the detection fibers distal to the probe tip and proximal to the spectrometer are arranged in a linear array at an end of a spectrometer leg of the bifurcated fiber.

In some instances, the one or more characteristics of the sample determined by the computing device include any of: an indication of whether the sample is natural or synthetic, or an indication of whether the sample is treated or untreated.

In another example embodiment, a method is provided. The method can include providing a laser device that includes multiple laser sources configured to excite a laser light and an optical combiner disposed adjacent to the multiple laser sources and configured to combine the excited laser light from the multiple laser sources and output incident light.

The method can also include disposing a cryochamber adjacent to the laser device. The cryochamber can be configured to receive a sample and the incident light output from the optical combiner. The incident light can be configured to interact with the sample, thereby producing responsive light. The method can also include connecting the cryochamber and the spectrometer via a bifurcated fiber including a probe tip to deliver the incident light to the cryochamber and receive the responsive light from the sample in response to the incident light to provide the responsive light to the spectrometer. The spectrometer can be configured to generate one or more measurement signals.

The method can also include electrically connecting a computing device to the spectrometer and a set of laser switches connected to any of the multiple laser sources. The computing device can be configured to cause the set of laser switches to individually control the excitation of the laser light from any of the multiple laser sources and to receive one or more measurement signals from the spectrometer to determine one or more characteristics of the sample.

In some instances, the computing device is further configured to initiate a sequence that causes the set of laser switches to individually control the excitation of the laser light from any of the multiple laser sources and thereby alter the incident light provided to the sample over a time period, and wherein the one or more measurement signals received at the spectrometer are configured to change during the time period.

In some instances, the one or more characteristics of the sample determined by the computing device include any of: an indication of whether the sample is natural or synthetic, or an indication of whether the sample is treated or untreated.

In another example, a non-transitory, computer-readable storage medium is provided. The non-transitory, computer-readable storage medium can have stored thereon a plurality of instructions, which, when executed by a processor of a computing device, cause the computing device to perform a series of steps. The steps can include instructing a set of laser switches to individually control laser light excitation from multiple laser sources that are part of a laser device. The laser device can include multiple laser sources and an optical combiner configured to combine the excited laser light from the multiple laser sources and output incident light. The incident light can be directed to a sample in a cryochamber, thereby producing responsive light. The steps can also include receiving one or more measurement signals from a spectrometer configured to generate one or more measurement signals from the responsive light. The steps can also include determining one or more characteristics of the sample based on one or more measurement signals.

In some instances, the instructions can further cause the computing device to instruct the set of laser switches to control laser light excitation from different combinations of the multiple laser sources according to a sequence over a time period, wherein the one or more measurement signals received at the spectrometer are configured to change during the time period.

In some instances, the one or more characteristics of the sample determined by the computing device include any of: an indication of whether the sample is natural or synthetic, or an indication of whether the sample is treated or untreated.

### BRIEF DESCRIPTION OF THE DRAWINGS

For a better understanding of the embodiments described in this application, reference should be made to the Detailed Description below, in conjunction with the following drawings in which like reference numerals refer to corresponding parts throughout the figures.

FIG. 1 is a block diagram of an example multi-laser spectroscopy system, according to some embodiments.

FIG. 2 is a block diagram of an example optical combiner, according to some embodiments.

FIG. 3A is a perspective view of an example sample chamber, according to some embodiments.

FIG. 3B is a perspective view of an example sample chamber with a bracket lowered onto a cryochamber housing to place a tip of a sample holder in contact with a probe tip in a cryochamber, according to some embodiments.

FIG. 4A is a top-view of an example cryochamber housing of the sample chamber, according to some embodiments.

FIG. 4B is a side view of the example cryochamber housing, according to some embodiments.

FIG. 5 is a perspective view of an example sample chamber, according to some embodiments.

FIG. 6 is a perspective view of a portion of an example sample chamber, according to some embodiments.

FIG. 7A is a side view of a bracket in an example sample chamber, according to some embodiments.

FIG. 7B is a top view of the bracket in an example sample chamber, according to some embodiments.

FIG. 8A is a side view of a sample holder, which may be used in a sample chamber as described in FIG. 1, according to some embodiments.

FIG. 8B is a side view of a sample holder, which may be used in a sample chamber as described in FIG. 3A, according to some embodiments.

FIG. 8C is a side view of a sample holder, which may be used in a sample chamber as described in FIG. 3B, according to some embodiments.

FIG. 8D is a side view of a sample holder, which may be used in a sample chamber as described in FIG. 5, according to some embodiments.

FIG. 9A is a first perspective view of an example sample holder that may be used with any sample chamber as described herein.

FIG. 9B is a second perspective view of an example sample holder that may be used with any sample chamber as described herein.

FIG. 10 is a side view of an example bifurcated fiber, according to some embodiments.

FIG. 11 is a flowchart illustrating a method of operating a multi-laser spectroscopy system, according to some embodiments.

FIG. 12 is a flowchart illustrating a method of operating a multi-laser spectroscopy system, according to some embodiments.

FIG. 13 is a flowchart illustrating a method of disposing a sample gemstone in contact with a probe tip within a cryochamber, according to some embodiments.

FIG. 14 is a diagram of an example networked system in accordance with certain aspects described herein.

FIG. 15 is a diagram of an example computer system in accordance with certain aspects described herein.

FIG. 16 is a block diagram of circuitry that, in some embodiments, may be used to implement various functions, operations, acts, processes, and/or methods disclosed herein.

### DETAILED DESCRIPTION OF EXAMPLE EMBODIMENTS

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the particular embodiments. In other instances, well-known data structures, timing protocols, software operations, procedures, and components have not been described in detail so as not to unnecessarily obscure aspects of the embodiments herein.

Systems and methods disclosed herein may enable a relatively low-cost and easy to use desktop cryogenic spectroscopy system. In comparison to other photoluminescence measurement devices that are large in space and require extensive trainings prior to operate, the systems as described herein can enable a desktop high resolution PL spectroscopy instrument that can perform in-situ measurement at cryogenic temperatures. Various examples disclosed herein may enable rapid, high-sensitivity and high-spectral-resolution cryogenic temperature PL and Raman measurements to be made over a broad spectral range, for example but not limited to, 400 – 1000 nm, using multiple laser excitations and close to real-time automated analysis of the results guiding the measurement sequence. Such a system may allow for simple control of the system in a production environment for repeated application with reliable results, thus increasing efficiency of both resources and time. In some example embodiments, robotic arms or robotic assemblies may be used instead of human users to load sample gemstones into systems disclosed herein.

The systems as described herein can provide for quicker testing to save time in analyzing each sample, as compared to testing times in other systems. The systems as described herein can also be less technically demanding in using in a production environment than other systems, resulting in increased efficiency in use and reduced resources needed to manufacture and maintain the components within the system. Such a system may also allow for sensitivity and spectral resolution to be increased, signal-to-noise ratio to be improved, general ease of use of the system by the operator to be improved, and system robustness and reliability to be improved. Safety may also be increased to reduce the exposure to laser light and liquid nitrogen for an operator. There can be a higher overall performance on a range of gemstone (e.g., diamond, color gemstone) types and sizes.

Systems described here may include a spectrometer arrangement, which may be switched on and initialized and calibrated, bringing components such as a charge coupled device (CCD) spectrometer to proper temperature. The multi-laser system may be used with the PL and Raman probe arrangement to analyze gemstones in the systems disclosed herein.

The systems and methods as described herein may also enable a more compact footprint for equipment than for other systems. For example, a multi-laser spectroscopy system may fit onto a standard desktop space of dimensions 60 by 30 inches or less, even when a relatively large, high-resolution, wide wavelength range spectrometer is used. In embodiments where a more compact, lower-resolution spectrometer is used, the multi-laser spectroscopy system may fit onto a desktop space of 30 by 15 inches or less.

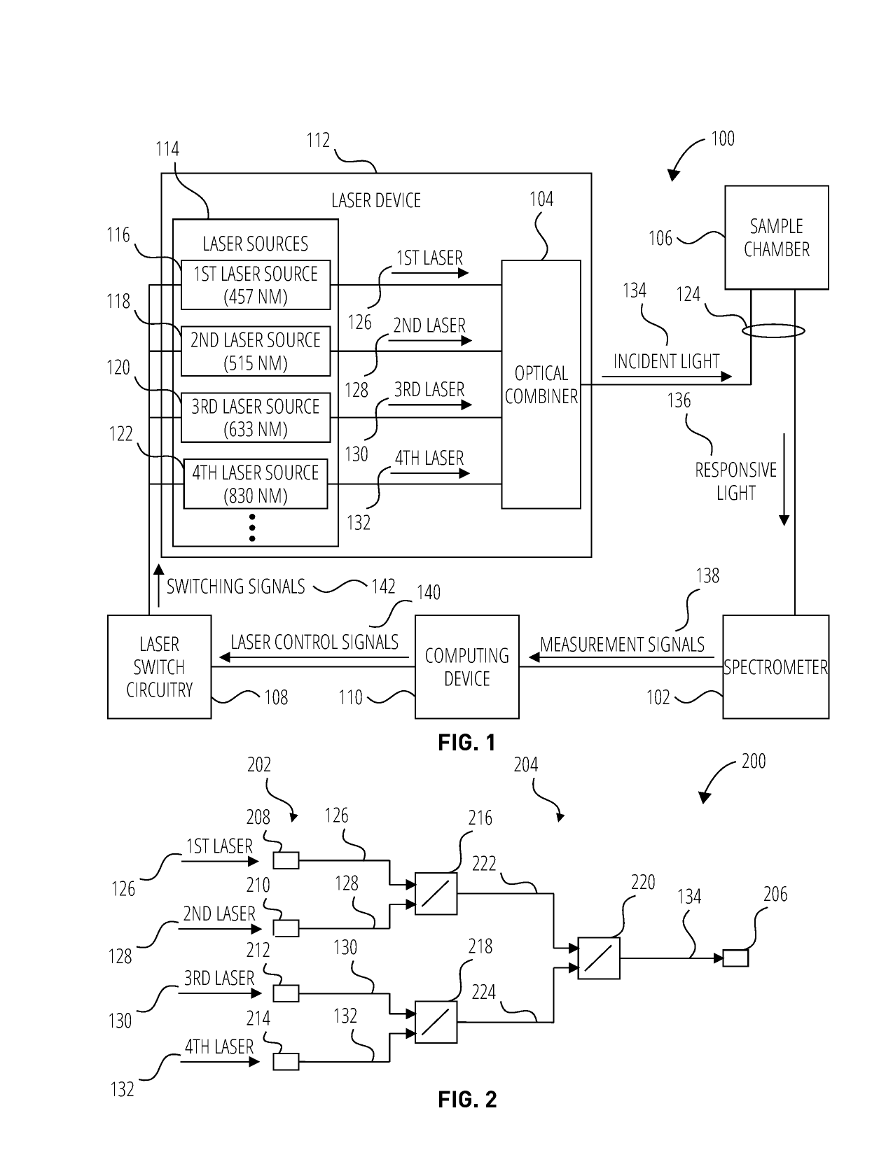

FIG. 1 is a block diagram of a multi-laser spectroscopy system 100, according to some embodiments. The multi-laser spectroscopy system 100 can include any of a spectrometer 102, a laser device 112, a sample chamber 106, a laser switch circuitry 108, and a computing device 110. Computing device 110 can be in electrical communication with the laser switch circuitry 108 and the spectrometer 102 (e.g., via electrical cabling, via wireless communication, etc.). The computing device 110 is configured to provide laser control signals 140 to the laser switch circuitry 108 to control switching on and off and/or to control power levels (e.g., intensities) of laser sources 114 of the laser device 112.

The laser switch circuitry 108 can be in electrical communication (e.g., wired and/or wireless electrical communication) with the laser sources 114. Upon receipt of the laser control signals 140 from computing device 110, the laser switch circuitry 108 can control the laser sources 114 according to the laser control signals 140 by providing switching signals 142 to the laser sources 114. The laser switch circuitry 108 may in this way modulate the output of each of the laser sources 114.

By way of non-limiting example, the laser switch circuitry 108 may include electrically controlled switches that individually and separately electrically connect the individual laser sources 114 to power. In such examples, the switching signals 142 may include power signals to power on and off the laser sources 114. When an individual switch of the laser switch circuitry 108 corresponding to an individual one of the laser sources 114 is turned off, the laser source associated with that switch is modulated to give zero output, which enables a user to selectively turn off any of the laser sources 114. Likewise, when an individual switch of the laser switch circuitry 108 corresponding to an individual one of the laser sources 114 is turned on, the laser source associated with that switch is modulated to give a non-zero output, which enables a user to selectively turn on any of the laser sources 114.

In this way, laser sources 114 may be independently modulated (switched on and off) via the laser switch circuitry 108, which may be configured for automated software control (e.g., via the laser control signals 140) and/or for manual control by a human operator. For example, the laser switch circuitry 108 may modulate the output of each of a number of the laser sources 114. In some examples, one, two, three or four lasers may be switched, with each laser having a different lasing wavelength. For gemstone studies laser excitations at frequencies corresponding to 457, 515, 633 and 830 nm wavelengths are particularly useful, but shorter and longer wavelength lasers may also be used in any combination or permutation.

In some examples, laser sources 114 include diode laser sources. In some examples, the laser sources 114 include a first laser source 116 that emits first laser light 126 with a center frequency corresponding to a wavelength of around 457 nm; a second laser source 118 that emits second laser light 128 with a center frequency corresponding to a wavelength of around 515 nm; a third laser source 120 that emits third laser light 130 with a center frequency corresponding to a wavelength of around 633 nm; and a fourth laser source 122 that emits fourth laser light 132 with a center frequency corresponding to a wavelength of around 830 nm. In the example of FIG. 1, laser sources 114 include four laser sources. In some examples, the number of laser sources may be greater than four (e.g., six or more laser sources) or less than four (e.g., one, two, or three laser sources).

In some examples, it may be useful to turn on more than one of the laser sources 114 at a time. In such examples, the laser control signals 140 may direct the laser switch circuitry 108 to turn on any number of the laser sources 114 at a given time. In some examples, it may be useful to turn on only one of the laser sources 114 at a time. In such examples, the laser control signals 140 may direct the laser switch circuitry 108 to turn on only one of the laser sources 114 at a time.

In some examples, one or more of the laser sources 114 may be replaced with a broadband light source such as a halogen lamp or light emitting diode (LED). In some such examples, the multi-laser spectroscopy system 100 may be used for absorbance measurements.

Laser sources 114 can be optically coupled to an optical combiner 104, which can deliver laser light received from the laser sources 114 to a common output as incident light 134. By way of non-limiting example, the optical combiner 104 may include multiple inputs, and an optical fiber or other suitable waveguide may optically connect each of the laser sources 114 to a different one of the inputs of the optical combiner 104.

In some examples, the optical combiner 104 may include an optical network to combine laser light from the laser sources 114 into incident light 134. For example, the optical combiner 104 may combine first laser light 126 from the first laser source 116, second laser light 128 from the second laser source 118, third laser light 130 from the third laser source 120, and fourth laser light 132 from the fourth laser source 122 into incident light 134. In this way, a single fiber connected between an exit port of the optical combiner 104 and the sample chamber 106 may transmit light from each of the laser sources 114, depending upon which of the lasers are modulated on by the laser control signals 140 and the switching signals 142 (e.g., one at a time, more than one at a time).

An optical output of the optical combiner 104 may be optically coupled to a bifurcated fiber 124, which may deliver the incident light 134 from the optical combiner 104 to the sample chamber 106. For example, the bifurcated fiber 124 may include a probe tip at the sample chamber 106 that delivers the incident light 134 to the sample chamber and collects responsive light 136 at the sample chamber 106 in response to incident light 134. The bifurcated fiber 124 also can provide the responsive light 136 to the spectrometer 102. The bifurcated fiber 124 can include a combination of fibers, some optically coupling the output of the optical combiner to the sample chamber 106 and others optically coupling the sample chamber to the spectrometer 102.

The spectrometer 102 may include components for measuring and/or analyzing spectral emissions (e.g., the responsive light 136) received from a sample gemstone loaded into the sample chamber 106 after excitation by incident light 134 from any of the laser sources 114. As discussed above, the spectrometer 102 may include or be in communication with one or more fiber lines of the bifurcated fiber 124 that run to the sample chamber 106 and terminates in a probe tip that may be placed in a cryochamber of the sample chamber 106. This probe tip may be in direct contact with a sample gemstone (e.g., with a table of the sample gemstone) loaded into the sample chamber 106.

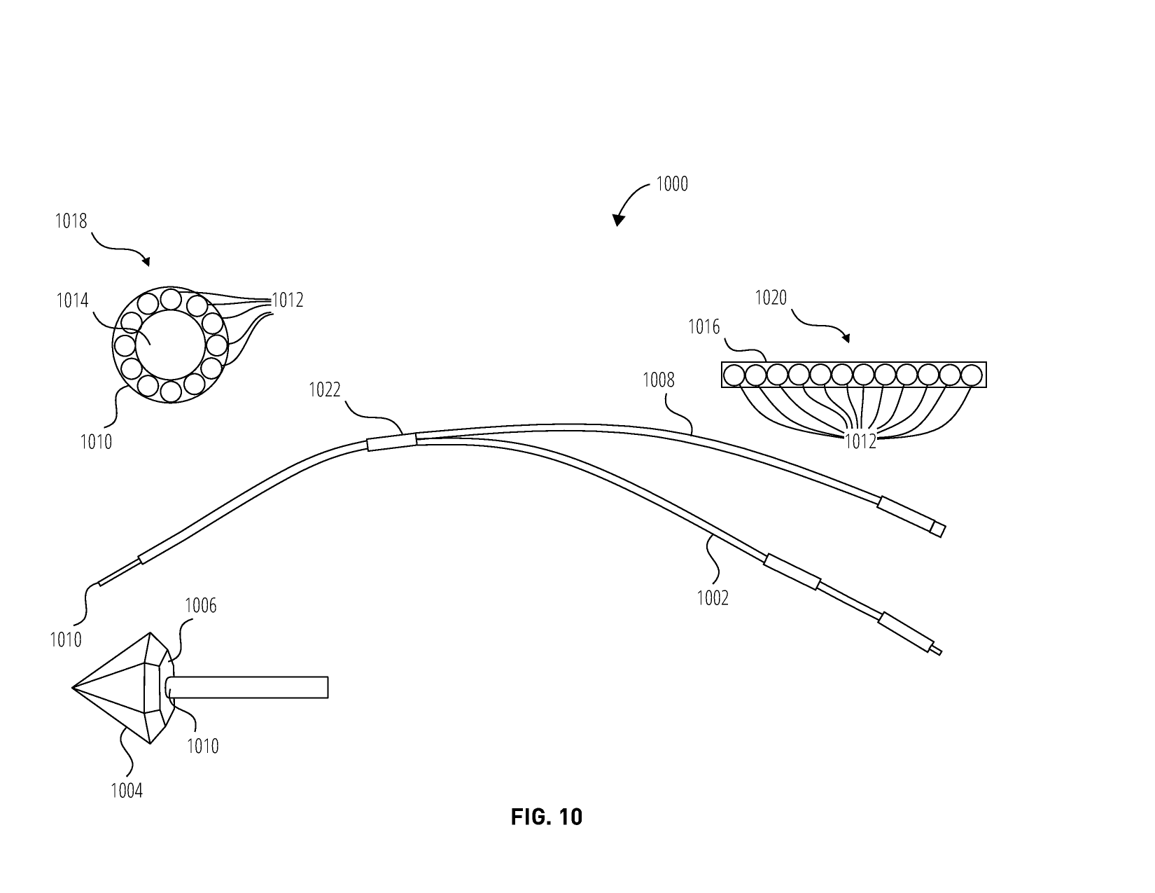

In some examples, the spectrometer 102 may be a high-resolution spectrometer 102 with software to enable full automation of the data collection over a broad wavelength range (for example but not limited to 400 nm to 1000 nm) and suitable for a production environment. In some examples, if a lower spectral resolution or narrower wavelength range meets a given specific need, the spectrometer 102 may be replaced by one or more very low-cost fixed grating CCD spectrometers. In these examples, the footprint and cost of the multi-laser spectroscopy system 100 may be reduced compared to that of the multi-laser spectroscopy system 100 including a more robust spectrometer. In such examples, a small compact spectrometer may be attached to the fiber. In some instances, less than a total number of fibers of the bifurcated fiber 124 that run between the spectrometer 102 and the sample chamber 106 may be used in these examples (e.g., less than all twelve detection fibers 1012 shown in FIG. 10).

The spectrometer 102 can be in electrical communication with the computing device 110 to deliver measurement signals 138 to the computing device 110. The spectrometer 102 may generate the measurement signals 138 corresponding to data regarding received signals (responsive light 136) from the gemstone sample in the sample chamber 106. The spectrometer 102 may send the measurement signals 138 to the computing device 110 for analysis and/or storage. In some instances, the computing device 110 may control aspects of the system 100, send and receive data including commands, obtain and display spectral data from spectrometer 102, etc. The computing device 110 can provide laser control signals 140 that causes the excitation of laser sources 114 according to a sequence that changes the excitation of different laser sources 114 during the sequence.

In some examples, the computing device 110 includes a user interface. In some examples, the computing device 110 is configured to send data including commands to the various components shown in FIG. 1, while also being receiving data from the various components in the system 100.

In operation, a user may load a sample gemstone into the sample chamber 106, bringing the sample gemstone into contact with a liquid nitrogen bath and the probe tip discussed above. The user may then interact with a user interface at the computing device 110 to start the multi-laser spectroscopy system 100 with a start button or software program initiated by the computing device 110. The software program may start the multi-laser spectroscopy system 100 by generating the laser control signals 140, and sending the laser control signals 140 to the laser switch circuitry 108. The laser switch circuitry 108 may activate one or more of the laser sources 114 to provide laser light to the optical combiner 104, which can combine the provided laser light into incident light 134. The bifurcated fiber 124 delivers the incident light 134 through the probe tip to the sample gemstone in the sample chamber 106 and collects responsive light 136. The bifurcated fiber 124 can deliver the responsive light 136 to the spectrometer 102. The spectrometer 102 can provide the measurement signals 138 to the computing device 110 for further analysis, such as to determine characteristics of the gemstone or to determine whether the gemstone is natural or synthetic, for example.

In some examples, the computer software executed by the computing device 110 may control switching on of the laser sources 114 one at a time, which may enable the computing device 110 to collect measurement signals 138 for the sample gemstone in the sample chamber 106 one at a time. Accordingly, the computing device 110 may accumulate separate measurement data corresponding to the measurement signals 138 for each of the laser sources 114 separately.

FIG. 2 is a block diagram of an optical combiner 200, which is an example of the optical combiner 104 of FIG. 1. The optical combiner 200 can include input ports 202, an optical network 204, and an output port 206. The input ports 202 can include a first input port 208, a second input port 210, a third input port 212, and a fourth input port 214 to receive the first laser light 126, the second laser light 128, the third laser light 130, and the fourth laser light 132, respectively, from the laser sources 114 of FIG. 1. Although the optical combiner 200 of FIG. 2 includes four input ports 202, the number of the input ports may be greater or less than four (e.g., to accommodate a number of the laser sources 114 of FIG. 1).

The optical network 204 includes dichroic filters to combine the input laser light into incident light 134 that may be provided to the bifurcated fiber 124 of FIG. 1 for delivery to the sample chamber 106. In the example illustrated in FIG. 2, the optical network 204 includes first dichroic beam splitter 216, second dichroic beam splitter 218, and third dichroic beam splitter 220.

The first dichroic beam splitter 216 can combine the first laser light 126 and the second laser light 128 into first sub-combination light 222, which includes the first laser light 126 and the second laser light 128. The second dichroic beam splitter 218 can combine the third laser light 130 and the fourth laser light 132 into second sub-combination light 224, which includes the third laser light 130 and the fourth laser light 132. The third dichroic beam splitter 220 can combine the first sub-combination light 222 and the second sub-combination light 224 into the incident light 134, which includes the first laser light 126, the second laser light 128, the third laser light 130, and the fourth laser light 132. In some examples, an optical combiner may include other types of optical combining elements such as fiber combiners, prism beam splitter combiners, or other optical combining elements in addition to, or instead of, dichroic beam splitters.

The optical network 204 provides the incident light 134 to the output port 206. The output port 206 may be in optical communication with the bifurcated fiber 124 of FIG. 1 (e.g., with one or more fibers of the bifurcated fiber 124 optically connecting the optical combiner 200 to the sample chamber 106 of FIG. 1). Accordingly, the optical combiner 104 may provide the incident light 134 including the first laser light 126, the second laser light 128, the third laser light 130, and the fourth laser light 132 to the sample chamber 106 via the bifurcated fiber 124.

In examples where a different number of input ports is included, an optical network may include a different number of dichroic beam splitters. An example optical combiner including two input ports may include an optical network including one dichroic beam splitter to combine two light beams (e.g., lasers) received at the respective input ports into a single light beam. An example optical combiner including eight input ports may include an optical network including seven dichroic beam splitters to combine eight light beams (e.g., lasers) received at the respective input ports into a single light beam.

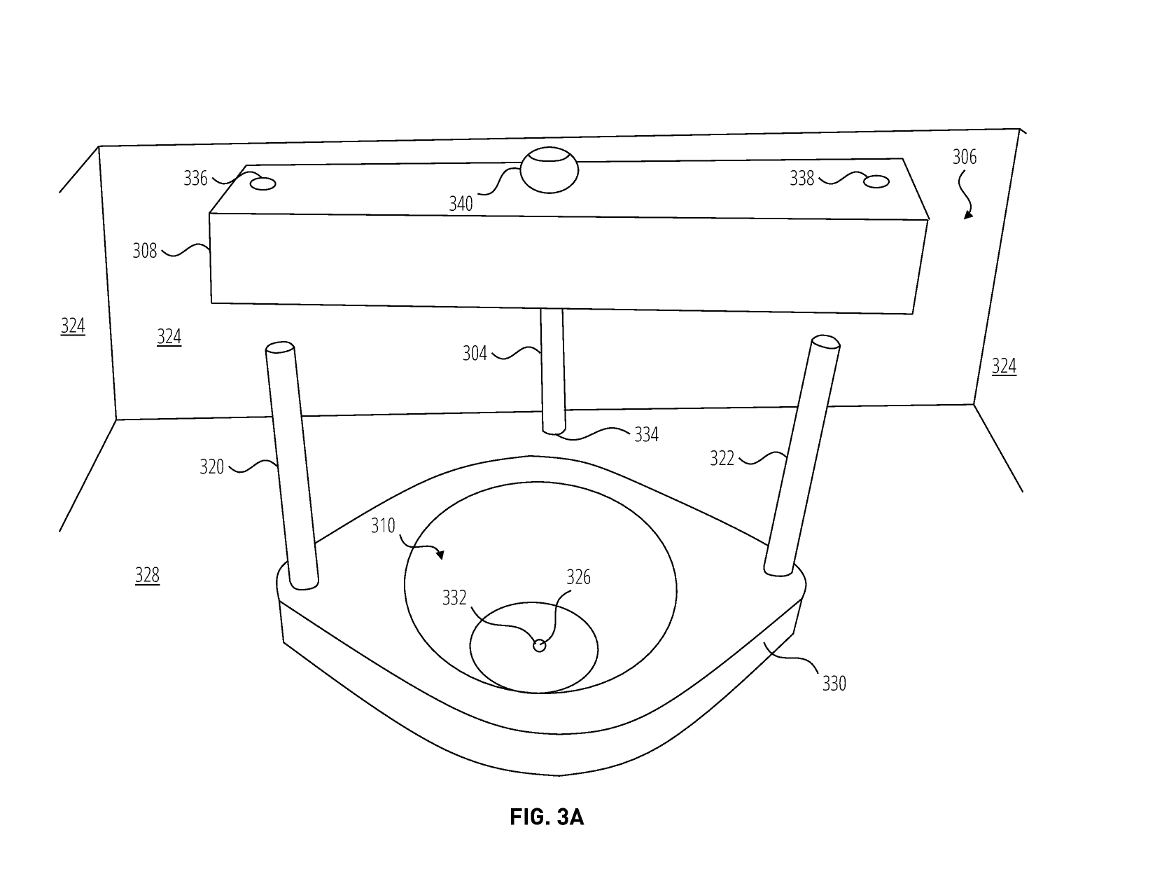

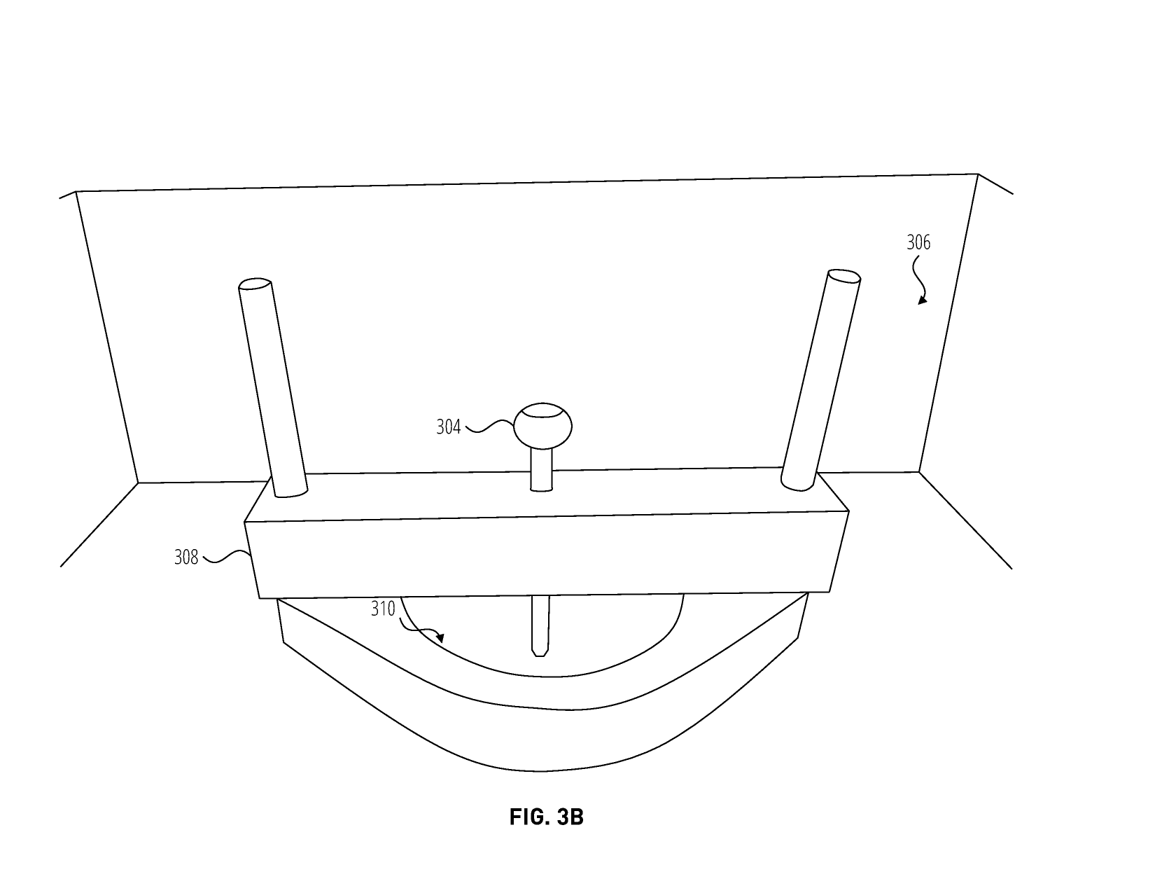

FIG. 3A is a perspective view of a sample chamber 306, which is an example of the sample chamber 106 of the multi-laser spectroscopy system 100 of FIG. 1. The sample chamber 306 is defined by enclosure walls 324 and a floor 328 to house components of the sample chamber 306. The sample chamber 306 may also include a top or a lid (not shown).

The sample chamber 306 can include a cryochamber housing 330, a probe tip 326 extending through a probe aperture 332 in the cryochamber housing 330, rails 320 and 322 extending from the cryochamber housing 330, a bracket 308, and a sample holder 304 extending through a holder passage through the bracket 308. In addition to the holder passage, the bracket 308 includes slots 336 and 338 extending therethrough to allow the rails 320 and 322 to extend through the bracket 308 at the slots 336, 338.

The sample holder 304 may be loaded with a sample gemstone at a tip 334 of the sample holder 304. With the sample holder 304 loaded with a sample gemstone at its tip 334, as the bracket 308 is lowered down along the rails 320 and 322, the sample gemstone may be aligned with the probe tip 326 in a bottom of a cryochamber 310 defined by the cryochamber housing 330. The sample gemstone may be placed in contact with the probe tip 326 as the bracket 308 is lowered all the way down onto the cryochamber housing 330.

In some examples, the sample holder 304 may be removable from the bracket 308 by lifting the sample holder 304 out through the holder passage. In some examples, the sample holder 304 may slide freely through the holder passage. In such examples, the sample holder 304 may be prevented from sliding all the way down through the holder passage by a knob 340, flange, or other structure having a diameter greater than that of the holder passage. In some examples, a diameter of the sample holder 304 may taper with a top portion having a diameter that is greater than a diameter of the holder passage and a lower portion near the tip 334 having a diameter that is less than the diameter of the holder passage. This tapered structure of the sample holder 304 may prevent the sample holder 304 from sliding down all the way through the bracket 308. The sample holder 304 may rest in the holder passage and hang down vertically below the bracket 308 but not slide all the way through and out of the bracket 308.

In some instances, a diameter of a body of the sample holder 304 may be between 7.16 millimeters (mm) to 7.22 mm. In some examples, the diameter of the body of the sample holder 304 may be between 6.5 mm and 8 mm. Such diameter may be correlated to a diameter of the holder passage that extends through the bracket 308 in order to allow the sample holder 304 to remain in position when loaded into the sample chamber 306. In some examples, a nub, ball, or other larger diameter top of the sample holder 304 (e.g., the knob 340) may also stop the sample holder 304 from sliding through the holder passage in the bracket 308, again with a larger diameter than the holder passage. In such a way, the sample holder 304 may be quickly removed, replaced, loaded and unloaded into the bracket 308 for sample testing.

In some examples, the holder 304 would not be removed from the bracket 308 while the sample gemstone is being mounted to the sample holder 304. For larger diameter sample gemstones (e.g., gemstone diameter greater than, equal to, or nearly as large as the diameter of the holder passage), a user may not be able to slide the holder through the holder passage if the sample gemstone was mounted to the sample holder 304 first.

With the sample holder 304 secured in place through the bracket 308 with a sample gemstone mounted to the tip 334 of the sample holder 304, the bracket 308 and sample holder 304 may be loaded into the sample chamber 306 for testing of the sample gemstone loaded into the sample holder 304. In some examples, the slots 336 and 338 through the bracket 308 near opposing ends of the bracket 308 enable the bracket 308 to slide over corresponding two rails 320 322 extending vertically from the cryochamber housing 330. In such an example, the rails 320 and 322 may be positioned on either side of a cryochamber 310 defined by the cryochamber housing 330. This arrangement allows for the sample gemstone to be centralized over a probe tip 326 within the cryochamber 310. Any arrangement of rails and corresponding holes may be used, for example, one, two, three, or four. In some examples, the cryochamber 310 may be loaded or filled with liquid nitrogen for better analyzing the sample gemstone in the sample holder 304. In such examples, the bracket 308 may simply slide down onto the top of the cryochamber housing 330 and rest on the two rails 320 and 322 while the sample holder 304 extends below the bracket 308 into the cryochamber 310. The length of the sample holder 304 and a depth of the cryochamber 310 may be matched such that the sample holder 304 hangs into the cryochamber 310, placing the sample gemstone in contact with the probe tip 326 positioned inside the cryochamber 310. In some examples, the length of the sample holder 304 may be about 4.44 to 4.50 inches long. In some examples, the length of the sample holder 304 may be between 3 and 5 inches long.

Liquid nitrogen may be disposed in the cryochamber 310. Also, the probe tip 326 may include one or more excitation fiber tips to deliver incident light (e.g., the incident light 134 of FIG. 1) to a sample gemstone deployed in the liquid nitrogen within the cryochamber 310. The probe tip 326 may also include one or more detection fiber tips to receive responsive light (e.g., the responsive light 136 of FIG. 1) and ultimately deliver the responsive light to a spectrometer (e.g., the spectrometer 102 of FIG. 1). In other words, the probe tip 326 may deliver incident light (e.g., the incident light 134 of FIG. 1) and receive emissions (e.g., the responsive light 136 of FIG. 1) from the sample gemstone. Measurement signals (e.g., the measurement signals 138 of FIG. 1) may be sent to a computer (e.g., the computing device 110 of FIG. 1) for analysis, according to various embodiments disclosed herein.

Within the sample chamber, liquid nitrogen may be loaded in order to submerge samples within the liquid nitrogen bath. This ensures that the sample gemstone is held at 77K (-196° C), which may ensure proper spectroscopy measurements of the sample gemstone. In such examples, enough liquid nitrogen may remain in liquid form for around 20 to 30 minutes before it evaporates. When the liquid nitrogen level becomes too low, a refill may ensure probe tips and sample gemstones are submerged for analysis. A user may check the level of liquid nitrogen by examining the level when a lid of the sample chamber 306 is open.

FIG. 3B is a perspective view of the sample chamber 306 of FIG. 3A with the bracket 308 lowered onto the cryochamber housing 330 to place the tip 334 of the sample holder 304 in contact with the probe tip 326 in the cryochamber 310. In FIG. 3B, the sample holder 304 is shown loaded into the bracket 308, which is shown loaded onto the two vertical rails 320 and 322, allowing the tip 334 of the sample holder 304 to extend down into the cryochamber 310 to expose any sample gemstone carried at the tip 334 to liquid nitrogen in the cryochamber 310. This arrangement also centers the sample holder 304 over the probe tip 326 for excitation and sampling according to various embodiments disclosed herein.

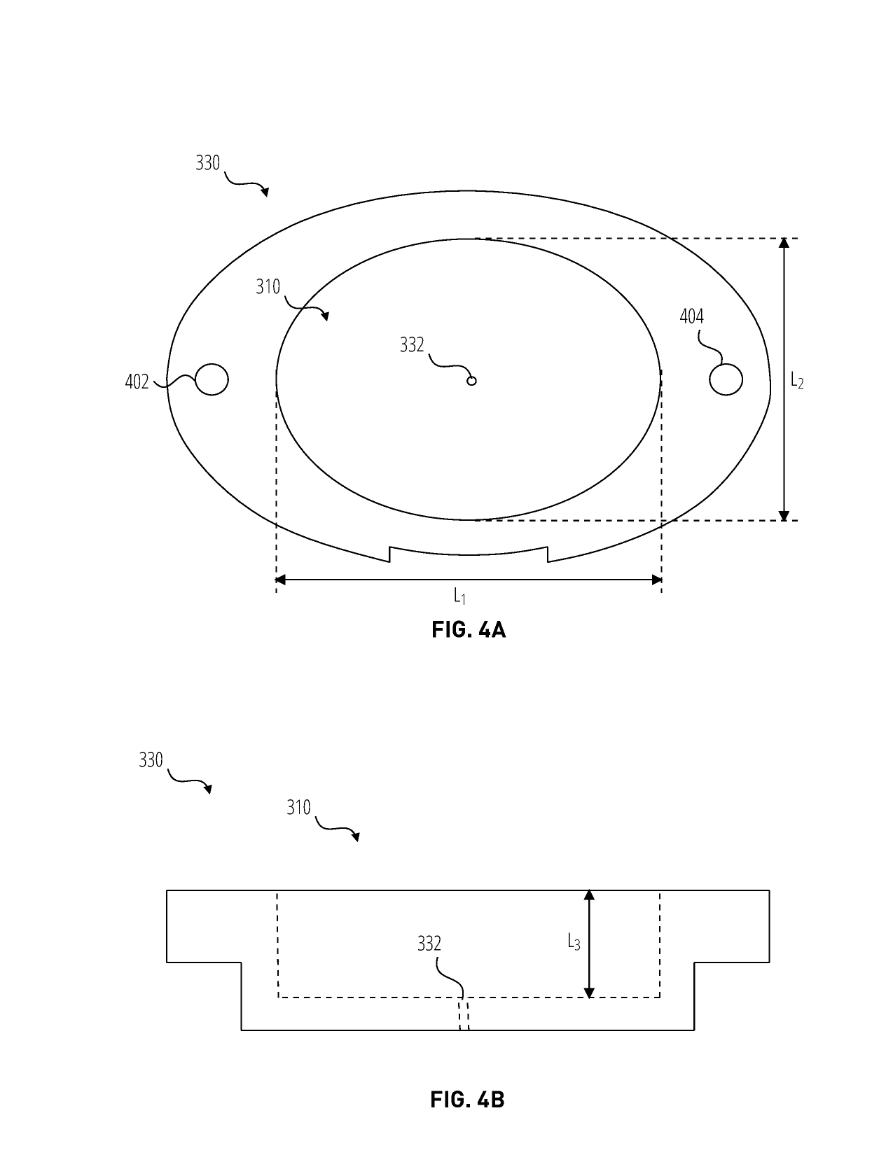

FIG. 4A is a top-view of the cryochamber housing 330 of the sample chamber 306 of FIGS. 3A and 3B. FIG. 4A illustrates the probe aperture 332 in the bottom of the cryochamber 310 and rail apertures 402 and 404 to support rails 320 and 322 at opposite sides of the cryochamber 310.In some examples, the cryochamber 310 in which the liquid nitrogen may be filled has an elliptical profile. In some examples, a longer dimension L₁ of the elliptical cryochamber 310 may be around 4.30 inches and a shorter dimension L₂ of the elliptical cryochamber 310 may be around 3.16 inches. In some examples, L₁ may be between 3 and 5 inches and L₂ may be between 2 and 4 inches.

FIG. 4B is a side view of the cryochamber housing 330 of FIG. 4A. FIG. 4B illustrates the probe aperture 332 extending through a bottom of the cryochamber housing 330 to enable a probe tip (e.g., the probe tip 326 of FIG. 1, the probe tip 1010 of FIG. 10) to extend therethrough into the cryochamber 310. A depth L₃ of the cryochamber 310 may be around 1.2 inches. In some examples, the depth L₃ may be between 0.5 and 1.5 inches. In some examples, the depth L₃ of the cryochamber 310 may be up to 2 inches.

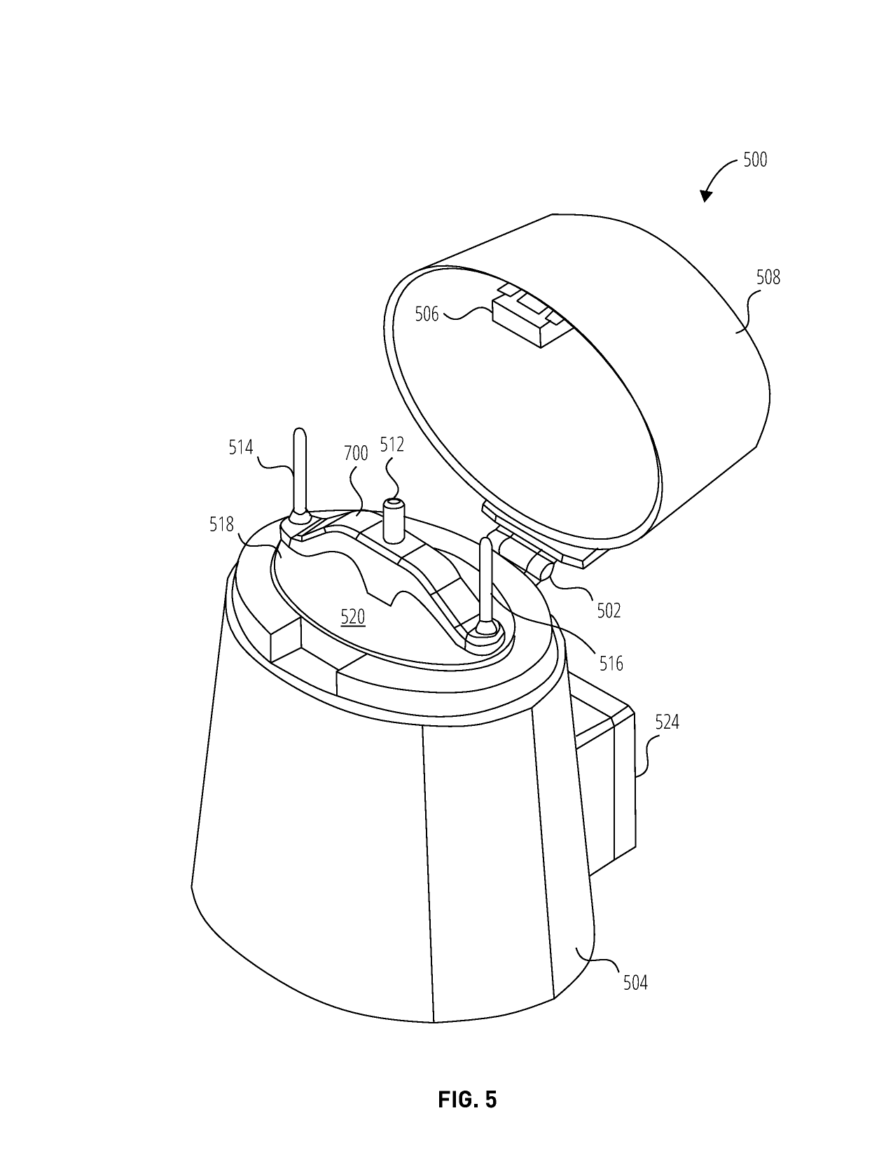

FIG. 5 is a perspective view of a sample chamber 500, which is another example of the sample chamber 106 of the multi-laser spectroscopy system 100 of FIG. 1. The sample chamber 500 includes a body 504 and a lid 508 mechanically connected to the body 504 by a hinge 502. In some examples, the body 504 may be made of or include foam. In some examples, the body 504 may be made of or include polymers or plastics. The sample chamber 500 also includes a cryochamber housing 518 defining a cryochamber 604 and including a cryochamber lid 520 to close the cryochamber 604. Closing the cryochamber 604 with the cryochamber lid 520 may thermally insulate liquid nitrogen in the cryochamber 604 from the ambient environment to slow conversion of the liquid nitrogen to a gas. The sample chamber 500 also includes rails 514 and 516 extending vertically from the cryochamber housing 518, as similarly discussed for the rails 320 and 322 extending from the cryochamber housing 330 of FIG. 3A and FIG. 3B.

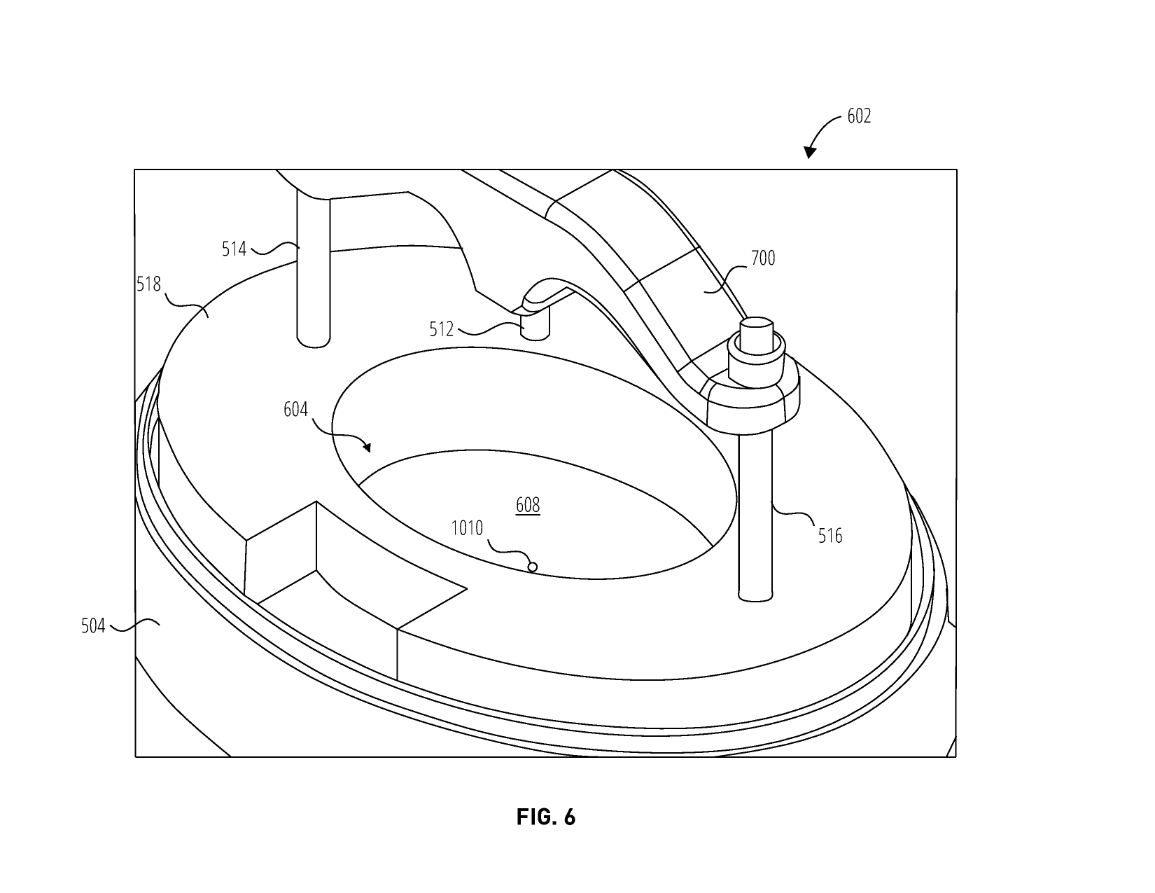

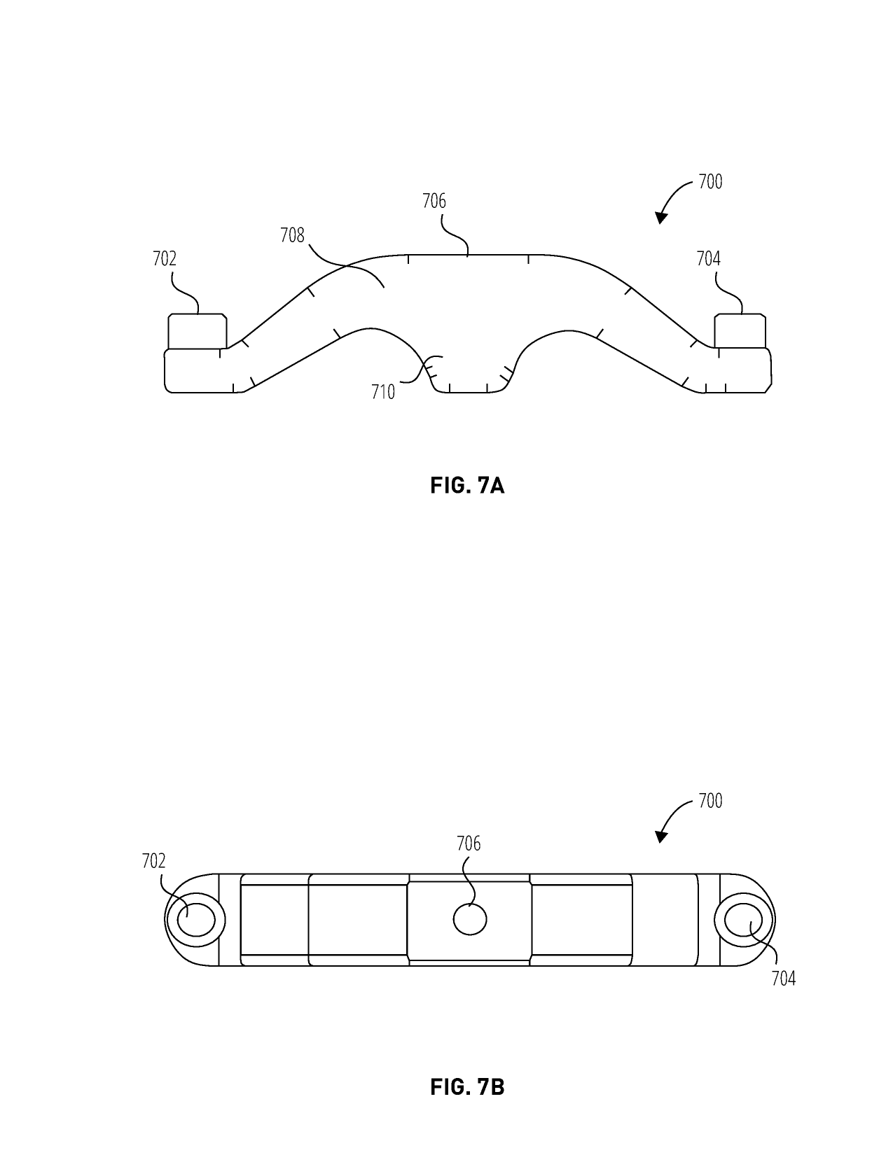

The sample chamber 500 also includes a sample holder 512 and a bracket 526. The sample holder 512 extends through the bracket 526 via a holder passage 706 (FIGS. 7A and 7B), as similarly discussed for the sample holder 304 and the bracket 308 of FIGS. 3A and 3B. The bracket also includes slots 702 and 704 (see FIGS. 7A and 7B) at opposing ends of the bracket 526, similar to the slots 336 and 338 of the bracket 308 of FIGS. 3A and 3B. The bracket 526 is slid down along the rails 514 and 516 to extend the sample holder 512 into the cryochamber 604, placing a sample gemstone at a tip (FIG. 6) of the sample holder 512 against a probe tip 1010 (FIG. 6) within the cryochamber (e.g., 604 shown in FIG. 6).

A user may open the sample chamber 500 by pivoting the lid 508 away from the body 504 to expose the cryochamber housing 518, the bracket 526, and the sample holder 512. A user may close the sample chamber 500 by pivoting the lid 508 back toward the body 504 and latching the lid 508 to the body 504 using a latch system 506. In some examples, the latch system 506 includes a magnetic interlock switch used to deactivate laser sources (e.g., the laser sources 114 of FIG. 1) when the lid 508 is opened. Accordingly, a technician may not be exposed to laser radiation when the lid 508 is opened. By way of non-limiting example, when the magnetic interlock is disengaged, a mechanical shutter may be activated at an exit port of the optical combiner 104 (FIG. 1), which may physically prevent laser radiation from entering the bifurcated fiber 124 (FIG. 1) and being delivered as the incident light 134 (FIG. 1) to the sample chamber 106 (FIG. 1).

The bracket 526 can be shaped to provide spaces between the bracket 526 and the cryochamber housing 518 to enable a user to insert fingers underneath the bracket 526 to more easily grip the bracket 526 while sliding the bracket 526 down or pulling the bracket 526 up along the rails 514 and 516 to more easily install and remove the bracket 526. In this way, a human user may handle, pick up, place down, or otherwise manipulate the bracket 526 while reducing the risk of user contact with freezing temperatures from liquid nitrogen in the cryochamber 604.

The sample chamber 500 also includes an enclosure 524 on a back side of the body 504. Enclosure 524 may be used to house one or more other components of a multi-laser spectroscopy system (e.g., the multi-laser spectroscopy system 100 of FIG. 1). By way of non-limiting example, the enclosure 524 may house the laser switch circuitry 108 of FIG. 1. The enclosure 524 contributes to the overall compactness of a multi-laser spectroscopy system.

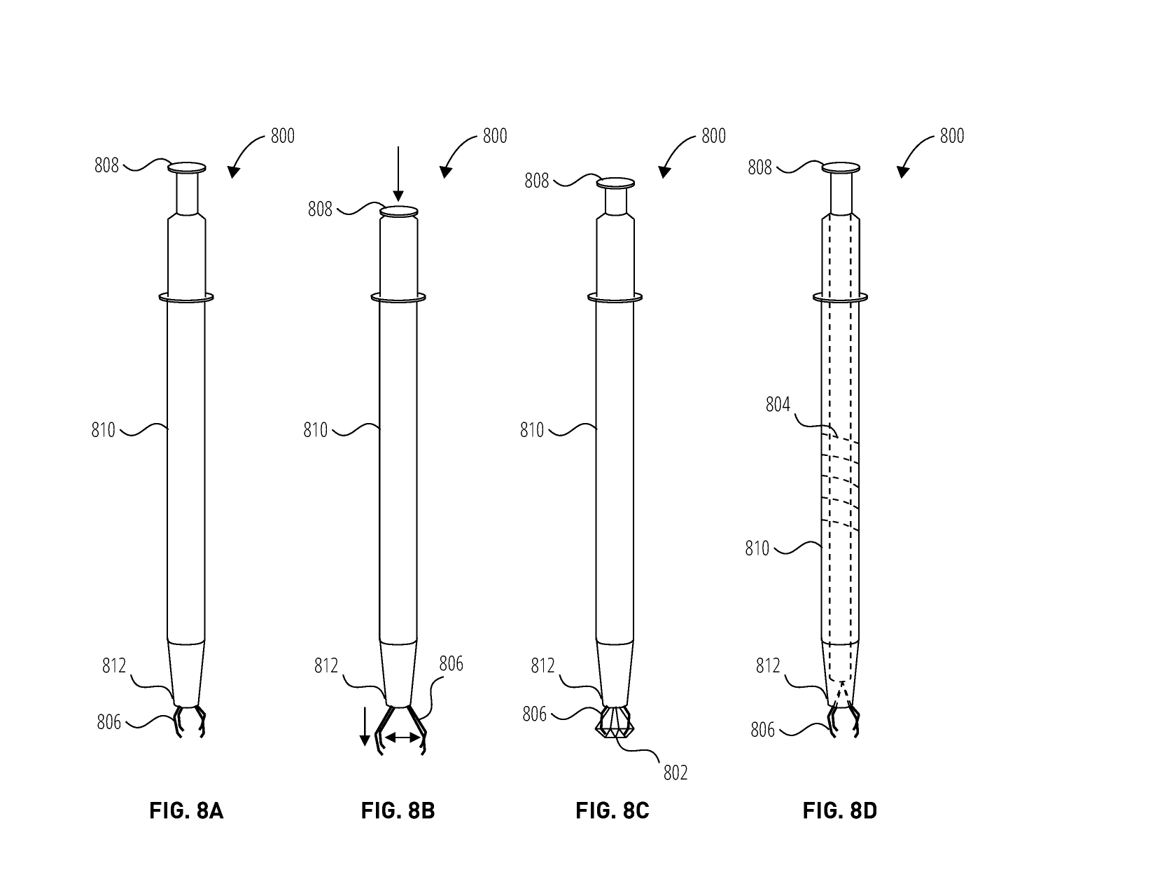

FIG. 6 is a zoomed-in perspective view of a portion 602 of the sample chamber 500 of FIG. 5. FIG. 6 shows the lid 508 removed from the cryochamber housing 518 to show the cryochamber 604 along with the probe tip 1010 at the bottom surface 608 of the cryochamber 604. In some examples, this probe tip 1010 sits proud of, or flush with, the bottom surface 608 of the cryochamber 604. In some examples, the probe tip 1010 sits just a few millimeters above the bottom surface 608 of the cryochamber 604. This design arrangement may ensure that a table of a sample gemstone is maintained in direct contact with the probe tip 1010 when loaded into the sample holder 512, even if the sample holder 512 includes holder arms 806 (FIGS. 8A-8D) that extend slightly below the table of the sample gemstone. In portion 602 of the sample chamber 500 shown in FIG. 6, the bracket 526 is pulled along the rails 514 and 516 away from the cryochamber housing 518.

In some examples, as shown in FIG. 8C, a sample gemstone 802 may be loaded to a tip of the gemstone holder 512 (as illustrated for the holder 800C of FIG. 8C) such that the gemstone table side is facing outward from the sample holder 512, to expose the table of the sample gemstone 802 to the probe tip 1010.

In some examples, the probe tip 1010 may be the tip of a bundle of optical fibers. The probe tip 1010 may be designed to maximize the optical signal and have a low thermal mass to reduce liquid nitrogen boil-off where the probe tip 1010 is in contact with the cryogen. This example design includes a probe tip 1010 with a small diameter in order to decrease its thermal mass and maximize the optical signal.

The probe tip 1010 would be positioned by sliding the probe tip 1010 through a hole in the cryochamber housing 518 with the hole diameter being such that a good friction fit is provided between the probe tip 1010 and the cryochamber housing 518 to prevent liquid nitrogen from leaking around the probe into the bottom of the sample chamber 500. In some examples, the cryochamber housing 518 is made of plastic or polymers. In some examples, the cryochamber housing 518 may be made of or include foam material with density characteristics that prevent leakage of the cryogen. Designs without foam fittings may utilize special grommets around the probe tip 1010 with additional thermal mass and complexity whereas the foam cryochamber housing 518 examples may not necessarily include such grommets. The foam cryochamber housing 518 examples may also enable simple removal and replacement of the probe tip 1010.

The sample holder 512 can be seen loaded into the bracket 700. When the sample holder 512 is loaded into the bracket 700 and then into the cryochamber 604 as shown in FIG. 5, the sample gemstone (e.g., the sample gemstone 802 of FIG. 8C) in the holder 512 may be positioned such that the sample gemstone (e.g., a table of the sample gemstone) contacts the probe tip 1010 when loaded into the cryochamber 604. In such examples, the rails 514, 516 and holder arms 806 (FIGS. 8A-8D) act to self-center the gemstone table over the probe tip 1010 at the bottom of the liquid nitrogen bath and in direct contact with the probe tip 1010. In such examples, no gap may form between the probe tip 1010 and the table side of the sample gemstone.

FIG. 7A is a side view of the bracket 526 of FIG. 5. FIG. 7B is a top view of the bracket 526 of FIG. 5. Referring to FIGS. 7A and 7B together, the bracket 526 may include two slots 702, 704 which may fit over the rails 514, 516 (FIGS. 5 and 6).

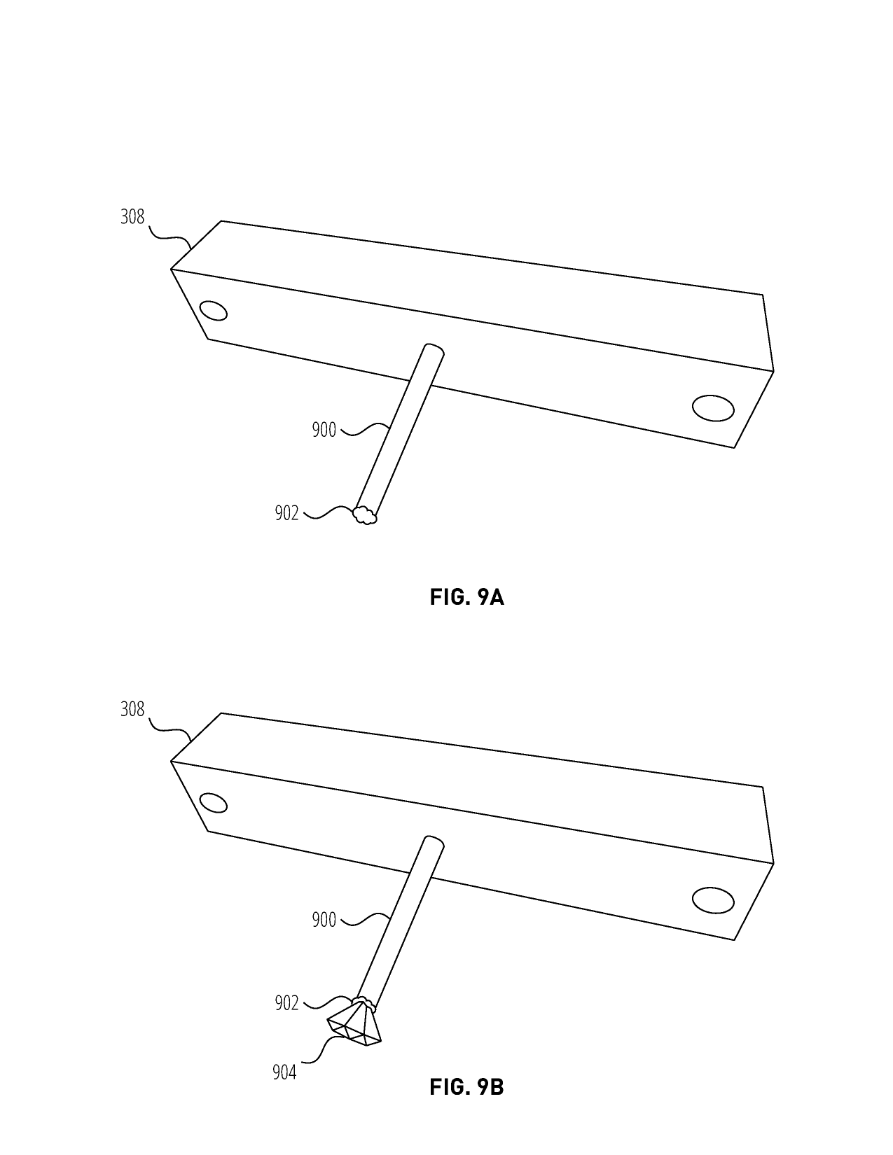

The bracket 526 can include an arched middle section 708 along with an extended portion 710 forming an arched T-shaped bracket to conform to hands and fingers of a user. This arrangement may allow for easy handling by an operator and also provide a longer holder passage 706 for supporting the holder (e.g., the holder 512 of FIG. 5, the holder 800A-D of FIGS. 8A-8D, the holder 900 of FIGS. 9A and 9B). In some examples, to allow a robotic arm assembly to grip and handle the bracket, slots, holes, indentations, or other features may be formed on or in the bracket 526 to allow a robotic arm to more easily handle the bracket 526. In some examples, the bracket 526 may have a square or rectangular cross-sectional shape (see the bracket 308 of FIGS. 3A and 3B).

In some examples, the bracket 526 may be around 6.5 inches long, 1 inch wide, and about 1.5 inches tall. In some examples, the holder passage 706 may have a diameter of around 21/64 inches to receive and secure the sample holder (e.g., the holder 512 of FIG. 5, the holder 800A-D of FIGS. 8A-8D, the holder 900 of FIGS. 9A and 9B). In some examples, the bracket 526 may be between around 4 and 7 inches long. In some examples, the bracket 526 may be between around 0.5 and 1.5 inches wide. In some examples, the bracket 526 may be around between 0.5 and 2.0 inches tall. Any ranges\ of bracket size that match up with the rails of the cryochamber may be used.

In some examples, the bracket 526 and/or the bracket 308 of FIGS. 3A and 3B may be made of thermally insulative material such as, but not limited to, polymers, ceramics, or other materials that thermally insulate from the cold temperature of the liquid nitrogen.

FIGS. 8A-8D are side views of a sample holder 800A-D, which may be used as the sample holder 304 in the sample chamber 306 of FIGS. 3A and 3B and/or as the sample holder 512 with the sample chamber 500 of FIG. 5. The holder 800A-D can include a plunger 808 at the top thereof, an elongated body 810, and a tip 812. The holder 800A-D also includes holder arms 806 extending from the tip 812. The holder arms 806 are configured for gripping a sample gemstone 802 at the tip 812 of the holder 800A-D, as illustrated in FIG. 8C. In some examples, the holder arms 806 include thin, flexible, wires, bent to grip and hold a sample gemstone 802.

FIGS. 8A and 8D illustrate the holder 800A-D with the plunger 808 in a non-depressed position. With the plunger 808 in the non-depressed position, the holder arms 806 remain in a non-extended position.

FIG. 8B can illustrate the holder 800B with the plunger 808 in a depressed position (e.g., pressed downward relative to the elongated body 810). With the plunger 808 in the depressed position, the holder arms 806 extend downward out of the tip 812 and outward into an extended position. With the holder arms 806 in the extended position, the holder arms 806 may receive a sample gemstone 802. For example, a user may depress the plunger 808, extending the holder arms 806 to open and accommodate the sample gemstone 802, place the sample gemstone 802 (e.g., with the table side down) into the holder arms 806, and release the plunger 808. As a result, the holder arms 806 may be drawn back or retracted upward into the tip 812 of the plunger 808, closing around and gripping the sample gemstone 802 into place at the tip 812, as illustrated in FIG. 8C. This secures the gemstone in the holder 800C and holds it steady in a table down configuration, ready to be loaded into a sample chamber

As illustrated in FIG. 8D, the holder 800D may include a mechanically resistive element (e.g., a spring 804) to bias the plunger 808 into the non-depressed position and the holder arms 806 into the non-extended position. The mechanically resistive element may also provide mechanical resistance against depression of the plunger 808 into the depressed position.

The holder 800A-D may be deployed through the holder passage (e.g., the holder passage through the bracket 308 of FIGS. 3A and 3B and/or the holder passage 706 of FIGS. 7A and 7B). With the holder 800A-D extending downward from the bracket and a sample gemstone secured to the tip 812 of the holder 800A-D, the bracket may be lowered over a cryochamber (e.g., the cryochamber 310 of FIGS. 3A and 3B, the cryochamber 604 of FIG. 6) to immerse the sample gemstone 802 in the liquid nitrogen and place a table of the sample gemstone 802 against the probe tip (e.g., the probe tip 326 of FIGS. 3A and 3B, the probe tip 1010 of FIG. 6).

FIGS. 9A and 9B are perspective views of another example sample holder 900 that may be used in the sample chambers 106, 306, and 500 of FIGS. 1, 3A and 3B and 5, respectively. FIGS. 9A and 9B illustrate the holder 900 extending through bracket 308 of FIGS. 3A and 3B. The sample holder 900 can include a low thermal mass straw with a small ball of reusable putty-like pressure-sensitive adhesive 902 placed at the tip. A sample gemstone 904 may be pressed into the adhesive 902 to hold the sample gemstone 904 in place, as illustrated in FIG. 9B. The holder 900 may be used to secure relatively smaller and fancy-shaped gemstones.

FIG. 10 is a side view of an example of a bifurcated fiber 1000 that may be used as the bifurcated fiber 124 of the multi-laser spectroscopy system 100 of FIG. 1. The bifurcated fiber 1000 includes a probe tip 1010, a bifurcation junction 1022, a spectrometer leg 1008, and a laser leg 1002. The bifurcated fiber 1000 also can include an excitation fiber 1014 and detection fibers 1012 in a probe tip side of the bifurcation junction 1022. The spectrometer leg 1008 includes the detection fibers 1012 and the laser leg 1002 includes the excitation fiber 1014.

In operation, light (e.g., the incident light 134 of FIG. 1) from an optical combiner (e.g., the optical combiner 104 of FIG. 1) travels down laser leg 1002 past the bifurcation junction 1022 and through the probe tip 1010 to a sample gemstone 1004 placed at the probe tip 1010 (e.g., in the sample chamber 106 of FIG. 1) and excites the sample gemstone 1004. A polished gemstone, such as a diamond, can be faceted so that most of the light that enters table 1006 of the sample gemstone 1004 is retroreflected back through table 1006. This property of polished gemstones is used to ensure that the light emitted from the sample gemstone 1004 due to the incident light 134 is passed back into the probe tip 1010, through the detection fibers 1012 (spectrometer leg 1008) into a spectrometer (e.g., the spectrometer 102 of FIG. 1).

In some examples, the detection fibers 1012 may surround the excitation fiber 1014 at the probe tip 1010. In some examples, the detection fibers 1012 may form a linear array 1016 of twelve fibers at the end of the spectrometer leg 1008 that connects to the spectrometer, as shown in a spectrometer leg end-on view 1020 of FIG. 10. In embodiments where the spectrometer includes a CCD spectrometer, this may increase the chances that height pixels on the CCD are utilized and also increases system sensitivity. In some examples, the detection fibers 1012 may be arranged in a flat linear array 1016 as shown in the spectrometer leg end-on view 1020 and then rearranged into the annular arrangement shown in the probe end-on view 1018 at the bifurcation junction 1022. The spectrometer relays the signal (e.g., measurement signals 138 of FIG. 1) to a computer (e.g., the computing device 110 of FIG. 1) for analysis.

The probe tip 1010 may include all the fibers (the excitation fiber 1014 and the detection fibers 1012) from both lines (the laser leg 1002 and the spectrometer leg 1008) as shown in a probe end-on view 1018 of FIG. 10. For example, the excitation fiber 1014 may be a central fiber and may have a diameter of around 700 micrometers. In some examples, the bifurcated fiber 1000 may include twelve detection fibers 1012 surrounding the excitation fiber 1014 at probe tip 1010. In some examples, a diameter of the detection fibers may be around 200 micrometers evenly spaced around the center fiber (excitation fiber 1014) in an annular fashion.

In operation, the excitation fiber 1014 extends from the laser device 112 (FIG. 1) and the detection fibers 1012 extend into the spectrometer 102 (FIG. 1). Other arrangements are contemplated within the scope of the disclosure, such as arrangements that include multiple excitation fibers 1014 and fewer detection fibers 1012, or one including a smaller diameter excitation fiber 1014. A larger diameter excitation fiber 1014 such as that illustrated in FIG. 10 is that the excitation fiber 1014 may also be used for making absorption measurements if the laser were swapped for an extended light source (such as an LED or halogen lamp).

The probe tip 1010 may be positioned inside a cryochamber (e.g., the cryochamber 310 of FIGS. 3A and 3B, the cryochamber 604 of FIG. 6) in order to position the probe tip 1010 proximate to (e.g., in contact with) the sample gemstone 1004, which may be loaded into a sample holder (e.g., the sample holder 304 of FIGS. 3A and 3B, the sample holder 512 of FIGS. 5 and 6).

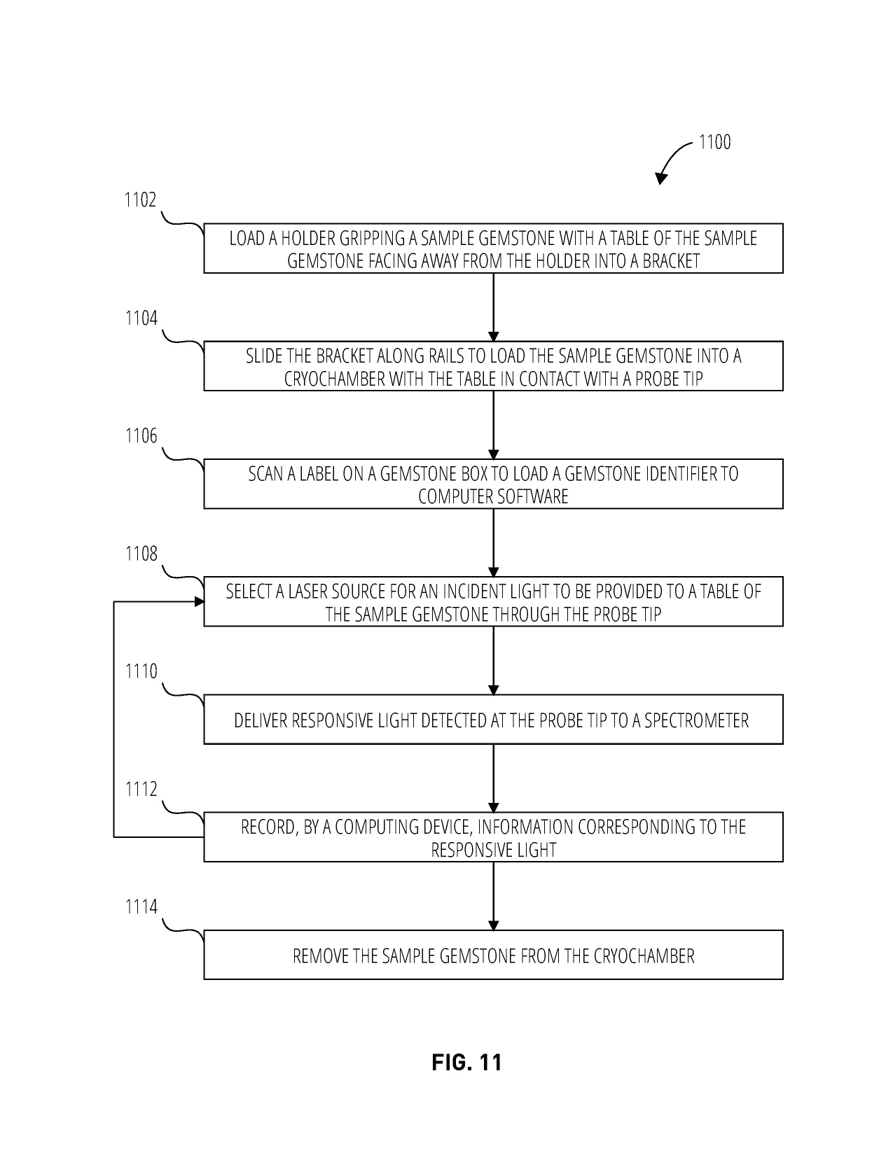

FIG. 11 is a flowchart illustrating a method 1100 of operating a multi-laser spectroscopy system (e.g., the multi-laser spectroscopy system 100 of FIG. 1), according to some embodiments. At operation 1102, the method 1100 includes loading a holder (e.g., any of the sample holders 304, 512, 800, 900 discussed above) gripping a sample gemstone with a table of the sample gemstone facing away from the holder into a bracket. For example, an operator may pick up a table side down gemstone in a spring-loaded holder (e.g., such as the holder 800 of FIGS. 8A-8D) mounted in a bracket (e.g., the bracket 308 of FIGS. 3A and 3B or the bracket 526 of FIGS. 5, 6, 7A and 7B) (e.g., through a holder passage such as the holder passage 706 of FIGS. 7A and 7B).

At operation 1104, the method 1100 includes sliding the bracket along rails (e.g., the rails 320 and 322 of FIGS. 3A and 3B, the rails 514 and 516 of FIGS. 5 and 6) to load the sample gemstone into a cryochamber (e.g., the cryochamber 310 of FIGS. 3A and 3B, the cryochamber 604 of FIG. 6) with the table in contact with a probe tip (e.g., the probe tip 326 of FIGS. 3A and 3B, the probe tip 1010 of FIGS. 6 and 10). For example, an operator may load the loaded holder and bracket into the cryochamber in the liquid nitrogen where the table of the gemstone touches the probe tip mounted in the cryochamber.

At operation 1106, the method 1100 includes scanning a label on a gemstone box to load a gemstone identifier to computer software (e.g., executed by the computing device 110 of FIG. 1). In this way, a computing device (e.g., the computing device 110 of FIG. 1) may store any analysis data with the identifier corresponding to the label on the gemstone box for future use, retrieval, and analysis.

At operation 1108, the method 1100 includes selecting a laser source (e.g., one of the laser sources 114 of FIG. 1) for an incident light (e.g., incident light 134 of FIG. 1) to be provided to a table of the sample gemstone through the probe tip. For example, the operator may select a first laser source 116 using a manual switch or by computer software. This selection commands laser switch circuitry 108 to allow energy from the first laser source 116 to be sent as incident light to the probe tip. The probe tip may deliver the incident light to the gemstone in the cryochamber.

At operation 1110, the method 1100 includes delivering responsive light (e.g., responsive light 136 of FIG. 1) detected at the probe tip to a spectrometer (e.g., the spectrometer 102 of FIG. 1). For example, the probe tip may collect the responsive light and may send the responsive light back to the spectrometer. At operation 1112, the method 1100 includes recording, by the computing device, information corresponding to the responsive light. For example, the spectrometer may send measurement signals (e.g., measurement signals 138 of FIG. 1) to the computing device. The computing device may record responsive signals for the selected laser source.

The method 1100 may include repeating operation 1108, operation 1110, and operation 1112 for each of the laser sources (e.g., for laser sources 114 including first laser source 116, second laser source 118, third laser source 120, and fourth laser source 122 of FIG. 1). In some examples, this analysis may take typically between 3 and 5 minutes per stone for 4 laser excitations, which may be approximately 1 minute per laser excitation.

At operation 1114, the method 1100 includes removing the sample gemstone from the cryochamber. For example, the operator may open the sample chamber (e.g., by opening the lid 508 of the sample chamber 500 of FIG. 5) and remove the holder with the gemstone and unload the gemstone that was just analyzed. In some examples, the operator may place the sample gemstone back into the gemstone box with the corresponding label.

Some examples, alone or in combination, include using a full software control of the lasers without any manual intervention needed for switching between them.

In some examples, alone or in combination, a measurement may be made by the system spectrometer with a first laser excitation and the automatically analyzed results of PL peak positions, full width at half maximum (FWHM) and intensities. Those measurements may be used to determine the parameters used for subsequent measurements with the same and/or alternative lasers. This process may be repeated at each subsequent laser excitation according to a pre-determined flow-chart built into the software. In this way, optimum data is collected for each sample gemstone without the need for any human intervention and may be automated by the system.

In some examples, alone or in combination, certain predictions as to the nature of the sample gemstone, based on the PL and Raman measurements, may be automatically generated. These predictions may include whether the sample is natural or synthetic, treated or untreated or may simply guide the operator as to which further measurements should be made on the sample in order to make an identification. The systems and methods disclosed herein may then be used for rapid, or close to real time, automated peak-fitting of the received Raman and/or PL spectra, which links into a measurement decision tree that determines the full range of automated measurements to be made in order to enable a decision on the nature of the gemstone under evaluation (natural or synthetic, treated or untreated, etc.).

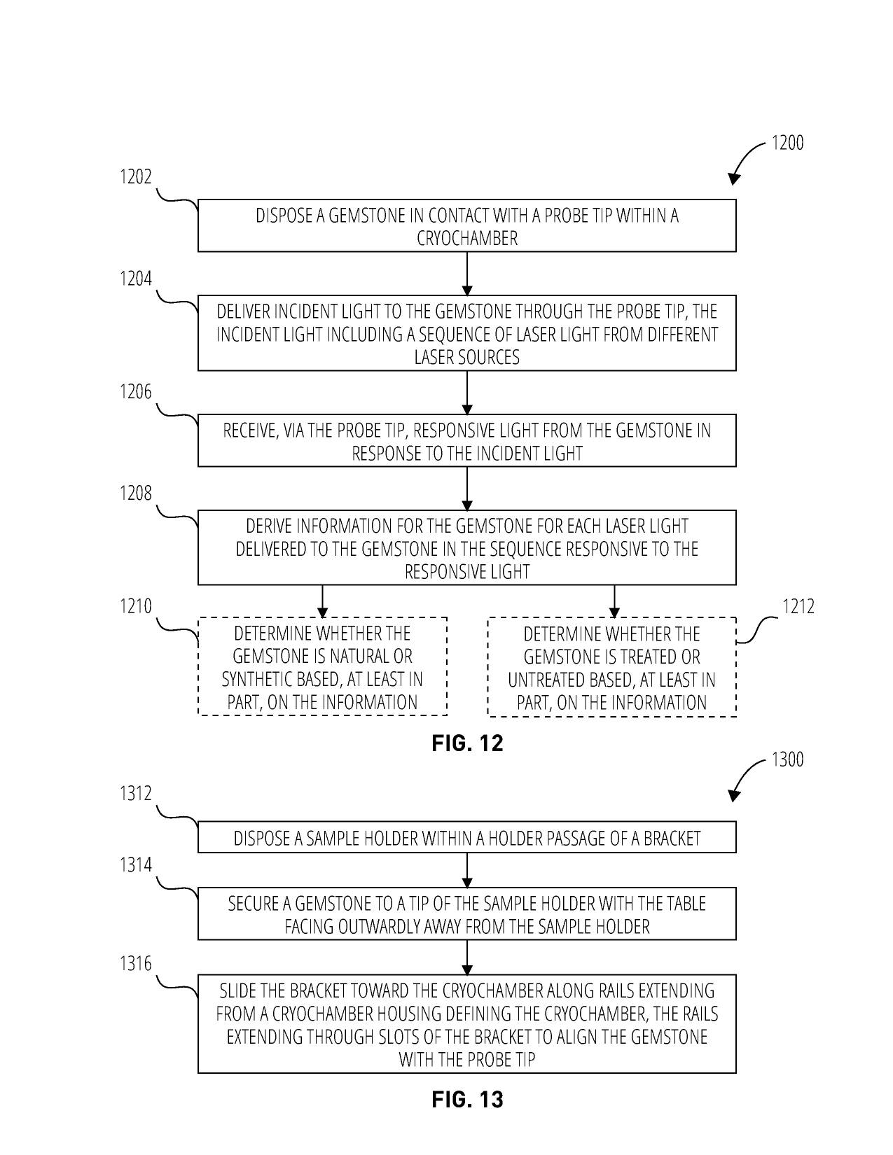

FIG. 12 is a flowchart illustrating a method 1200 of operating a multi-laser spectroscopy system (e.g., the multi-laser spectroscopy system 100 of FIG. 1), according to some embodiments. At operation 1202 the method 1200 includes disposing a gemstone in contact with a probe tip (e.g., the probe tip 326 of FIGS. 3A and 3B, the probe tip 1010 of FIGS. 6 and 10) within a cryochamber (e.g., the cryochambers 310 of FIGS. 3A and 3B, the cryochamber 604 of FIG. 6). An example of a method 1300 of disposing the gemstone in contact with the probe tip within the cryochamber is discussed in more detail below with reference to FIG. 13.

At operation 1204, the method 1200 includes delivering incident light (e.g., the incident light 134 of FIG. 1) to the gemstone through the probe tip, the incident light including a sequence (e.g., a one at a time sequence) of laser light from different laser sources (e.g., the laser sources 114 of FIG. 1). In some examples, the different laser sources include four laser sources including a first laser source (e.g., the first laser source 116 of FIG. 1), a second laser source (e.g., the second laser source 118 of FIG. 1), a third laser source (e.g., the third laser source 120 of FIG. 1), and a fourth laser source (e.g., the fourth laser source 122 of FIG. 1).

At operation 1206, the method 1200 includes receiving, via the probe tip, responsive light (e.g., the responsive light 136 of FIG. 1) from the gemstone in response to the incident light. In some embodiments,

At operation 1208, the method 1200 includes deriving information for the gemstone for each laser light delivered to the gemstone in the sequence in response to the responsive light. In some embodiments, the information may be provided, by a spectrometer (e.g., the spectrometer 102 of FIG. 1), to a computing device (e.g., the computing device 110 of FIG. 1) as measurement signals (e.g., the measurement signals 138 of FIG. 1). In some embodiments, deriving the information may include measuring the response light the with the spectrometer, which may be optically coupled to the probe tip via one or more detection fibers (e.g., the detection fibers 1012 of FIG. 10). The computing device may use the information to determine one or more parameters of the gemstone. For example, at operation 1210, the method 1200 may include determining whether the gemstone is natural or synthetic based, at least in part, on the information. As another example, at operation 1212, the 1200 may include determining whether the gemstone is treated or untreated based, at least in part, on the information.

FIG. 13 is a flowchart illustrating a method 1300 of disposing a gemstone in contact with a probe tip within a cryochamber, according to some embodiments. At operation 1312, the method 1300 includes disposing a sample holder (e.g., the sample holder 304 of FIGS. 3A and 3B, the sample holder 512 of FIG. 5 and FIG. 6, the sample holder 800A-D of FIGS. 8A-8D, the sample holder 900 of FIGS. 9A and 9B) within a holder passage of a bracket (e.g., the bracket 308 of FIGS. 3A and 3B, the bracket 526 of FIGS. 5, 6, 7A and 7B). At operation 1314, the method 1300 includes securing the gemstone to a tip of the sample holder with the table facing outwardly away from the sample holder. At operation 1316, the method 1300 includes sliding the bracket toward the cryochamber along rails (e.g., the rails 320 and 322 of FIGS. 3A and 3B, the rails 514 and 516 of FIGS. 5 and 6) extending from a cryochamber housing (e.g., the cryochamber housing 330 of FIGS. 3A-4B, the cryochamber housing 518 of FIGS. 5 and 6) defining the cryochamber, the rails extending through slots (e.g., the slots 336 and 338 of FIGS. 3A-4B, the slots 702 and 704 of FIGS. 7A and 7B) of the bracket to align the gemstone with the probe tip.

In some embodiments, a method of operating a multi-laser spectroscopy system disposing a gemstone in contact with a probe tip within a cryochamber. The method can also include delivering incident light to the gemstone through the probe tip, the incident light including a sequence of laser light from different laser sources. The method can also include receiving, via the probe tip, responsive light from the gemstone in response to the incident light. The method can also include deriving information for the gemstone for each laser light delivered to the gemstone in the sequence responsive to the responsive light.

In some instances, disposing the gemstone in contact with the probe tip within the cryochamber comprises disposing a table of the gemstone in contact with the probe tip.

In some instances, disposing the table of the gemstone in contact with the probe tip comprises disposing a sample holder within a holder passage of a bracket, securing the gemstone to a tip of the sample holder with the table facing outwardly away from the sample holder, and sliding the bracket toward the cryochamber along rails extending from a cryochamber housing defining the cryochamber, the rails extending through slots of the bracket to align the gemstone with the probe tip.

In some instances, delivering the incident light to the gemstone includes turning the different laser sources on one at a time and delivering the laser light to the probe tip through an optical combiner.

In some instances, the method can include determining whether the gemstone is natural or synthetic based, at least in part, on the information.

In some instances, the method can include determining whether the gemstone is treated or untreated based, at least in part, on the information.

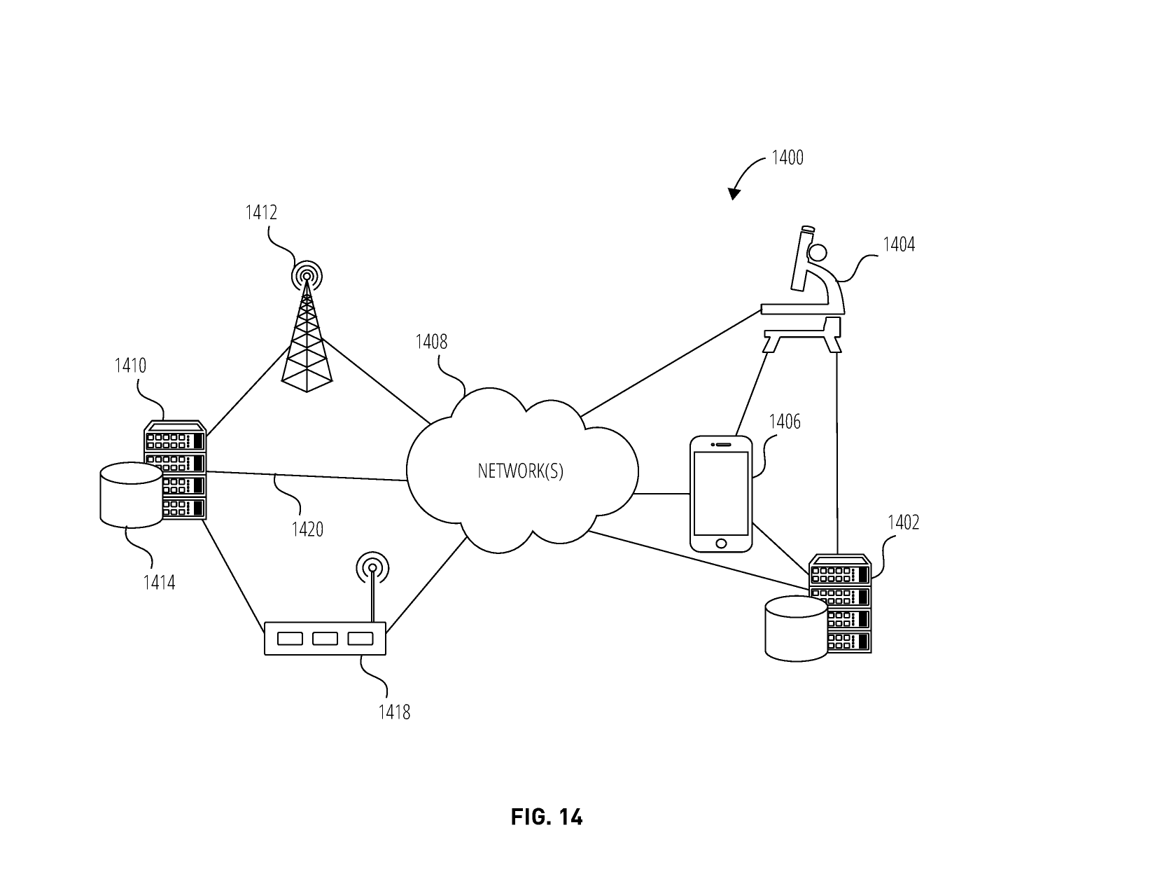

Various systems and methods disclosed herein may utilize a networked computing arrangement 1400 as shown in FIG. 14. The networked computing arrangement 1400 includes a multi-laser spectroscopy system 1404 (e.g., the multi-laser spectroscopy system 100 of FIG. 1) and a computer 1402 (e.g., the computing device 110 of FIG. 1). The computer 1402 may be used to process the data of the spectrometer (e.g., the measurement signals 138 of FIG. 1). The computer 1402 used for these operations may be any number of kinds of computers such as those included in the components, and/or another computer arrangement in communication with the components including but not limited to a laptop, desktop, tablet, phablet, smartphone 1406, or any other kind of device used to process and transmit digitized data.

Referring once again to FIG. 14, the data captured for calibration file, stone sample identifying information, and/or location, from whichever computer 1402 may be analyzed on a back-end computer 1410 instead of or in addition to a local computer (e.g., the computer 1402). In such examples, data may be transmitted to a back-end computer 1410 and associated data storage 1414 via one or more network(s) 1408 for saving, analysis, computation, comparison, or other manipulation. In some examples, additionally or alternatively, the transmission of data may be wireless by cellular data networks 1412 or via Wi-Fi transmission with associated routers and hubs 1418. In some examples, additionally or alternatively, the transmission may be through a wired connection 1420. In some examples, additionally or alternatively, the transmission may be through a network such as the internet to the back-end computer 1410 and associated data storage 1414.

At the back-end computer 1410 and associated data storage 1414, the spectrometer data, sample identification, sample location, time, and/or date may be stored, analyzed, compared to previously stored image data for matching, identification, and/or any other kind of data analysis. In some examples, additionally or alternatively, the storing, analyzing, and/or processing of data may be accomplished at the computer 1402, which may be involved in the original data collection. In some examples, additionally or alternatively, the data storing, analyzing, and/or processing may be shared between the local computer 1402 and a back-end computer 1410. In such examples, networked computer resources may allow for more data processing power to be utilized than may be otherwise available at the local computers 1402. In such a way, the processing and/or storage of data may be offloaded to the compute resources that are available. In some examples, additionally or alternatively, the networked computer resources may be virtual machines in a cloud or distributed infrastructure. In some examples, additionally or alternatively, the networked computer resources may be spread across multiple physical or virtual computer resources by a cloud infrastructure. The example of a server implemented as a single back-end computer 1410 is not intended to be limiting and is only one example of a compute resource that may be utilized by the systems and methods described herein. In some examples, additionally or alternatively, artificial intelligence and/or machine learning may be used to analyze the spectroscopic data from the samples. Such systems may employ data sets to train algorithms to help produce better and better results.

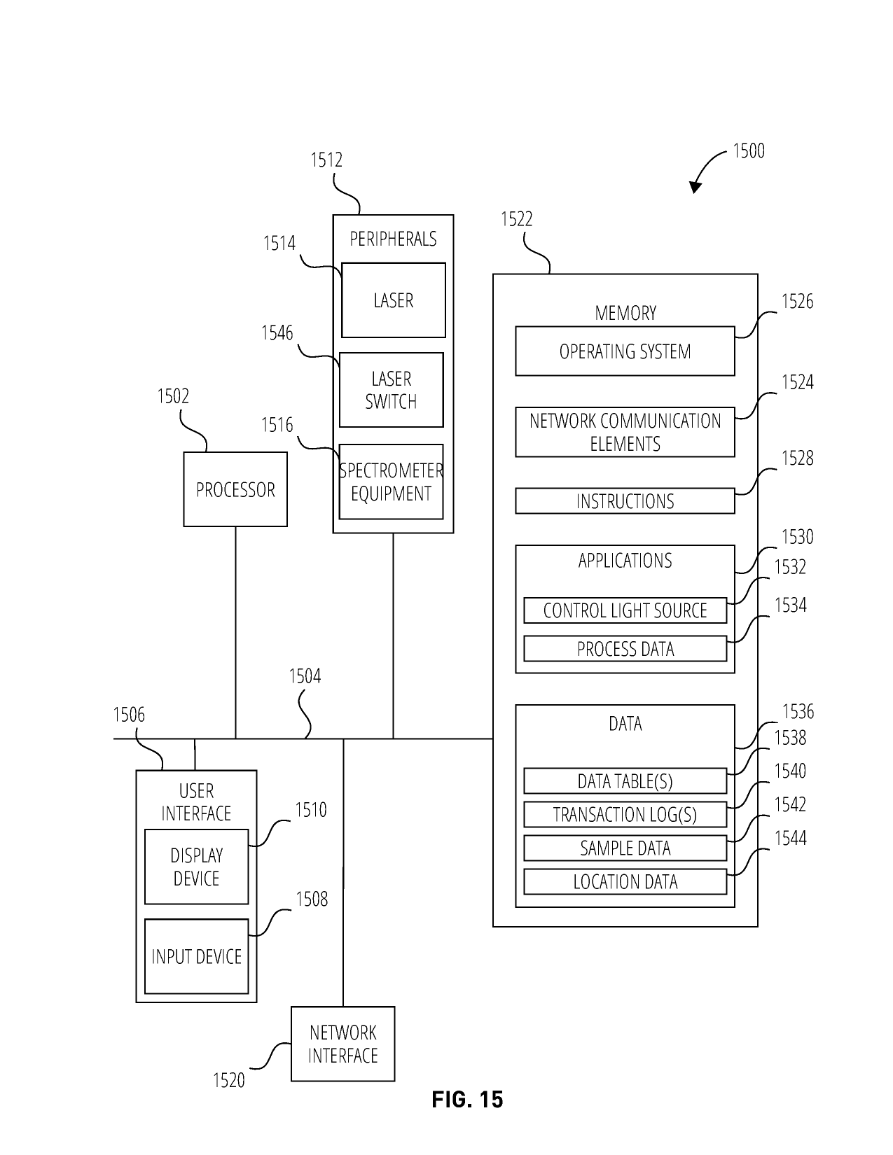

FIG. 15 is a block diagram of an example computing system 1500 that may be used in systems and methods disclosed herein. The computing system 1500 may be an example of the computing device 110 of FIG. 1. The example computing system 1500 includes a central processing unit (CPU) or processor 1502 in communication, by communication elements 1504 (e.g., a bus or other communication elements), with a user interface 1506. The user interface 1506 includes an example input device 1508 such as a keyboard, mouse, touchscreen, button, joystick, or other user input device(s). The user interface 1506 also includes a display device 1510 such as a screen. The communication elements 1504 are in communication with the processor 1502 and other components. The network interface 1520 may allow the computing system 1500 to communicate with other computers, databases, networks, user devices, or any other computing capable devices. In some examples, additionally or alternatively, the method of communication may be through WIFI, cellular, Bluetooth Low Energy, wired communication, or any other kind of communication. In some examples, additionally or alternatively, the example computing system 1500 includes peripherals 1512 also in communication with the processor 1502. For example, the peripherals 1512 may include lasers 1514 (e.g., the laser sources 114 of FIG. 1), a laser switch 1546 (e.g., the laser switch circuitry 108 of FIG. 1), and spectrometer equipment 1516.

In some examples, computing system 1500 includes a memory 1522 in communication with the processor 1502. In some examples, additionally or alternatively, this memory 1522 may include instructions to execute software such as an operating system 1526, network communication elements 1524, other instructions 1528, applications 1530 (applications to control light sources 1532, applications to process data 1534 such as image pixels), data 1536 (e.g., data tables 1538, transaction logs 1540, sample data 1542, sample location data 1544, or any other kind of data).

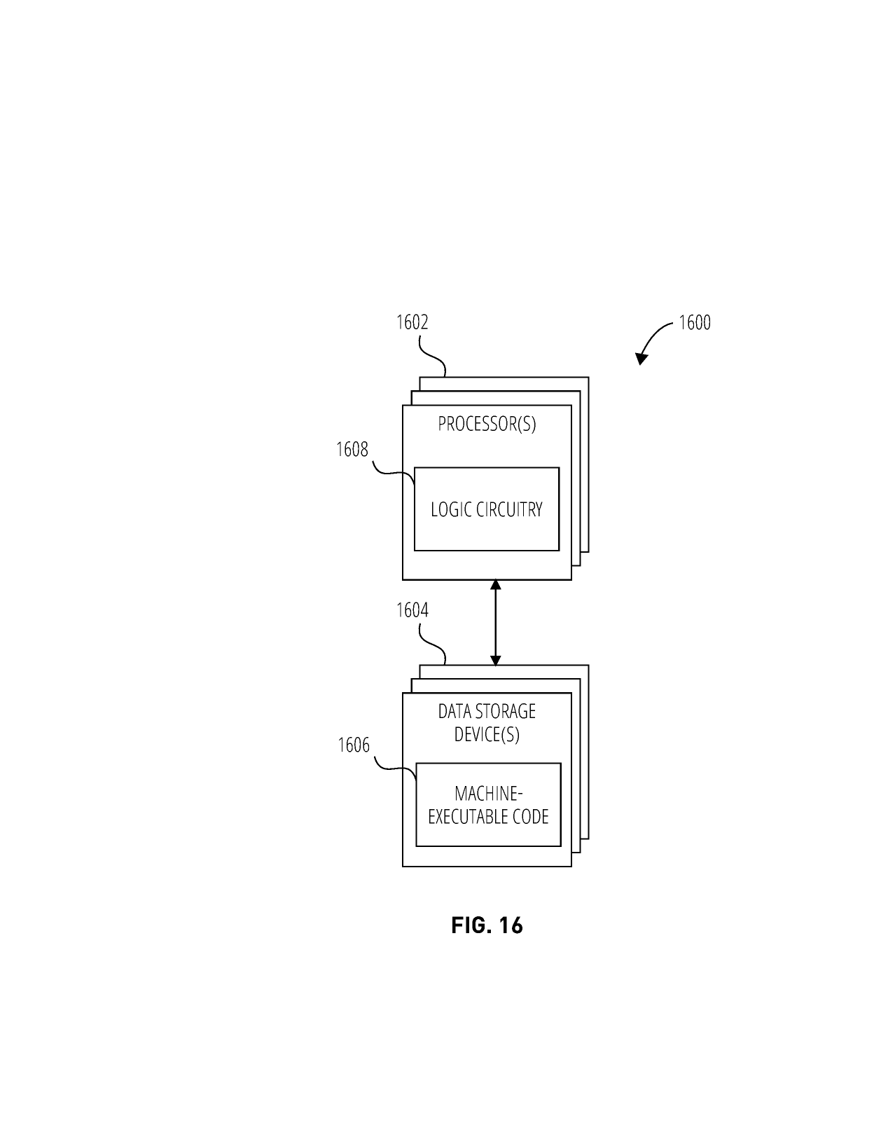

It will be appreciated by those of ordinary skill in the art that functional elements of embodiments disclosed herein (e.g., functions, operations, acts, processes, and/or methods) may be implemented in any suitable hardware, software, firmware, or combinations thereof. FIG. 16 illustrates non-limiting examples of implementations of functional elements disclosed herein. In some embodiments, some or all portions of the functional elements disclosed herein may be performed by hardware specially configured for carrying out the functional elements.

FIG. 16 is a block diagram of circuitry 1600 that, in some embodiments, may be used to implement various functions, operations, acts, processes, and/or methods disclosed herein. The circuitry 1600 includes one or more processors 1602 (sometimes referred to herein as “processors 1602”) operably coupled to one or more data storage devices (sometimes referred to herein as “storage 1604”). The storage 1604 includes machine-executable code 1606 stored thereon and the processors 1602 include logic circuitry 1608. The machine-executable code 1606 includes information describing functional elements that may be implemented by (e.g., performed by) the logic circuitry 1608. The logic circuitry 1608 is adapted to implement (e.g., perform) the functional elements described by the machine-executable code 1606. The circuitry 1600, when executing the functional elements described by the machine-executable code 1606, should be considered as special purpose hardware configured for carrying out functional elements disclosed herein. In some embodiments the processors 1602 may be configured to perform the functional elements described by the machine-executable code 1606 sequentially, concurrently (e.g., on one or more different hardware platforms), or in one or more parallel process streams.

When implemented by logic circuitry 1608 of the processors 1602, the machine-executable code 1606 is configured to adapt the processors 1602 to perform operations of embodiments disclosed herein. For example, the machine-executable code 1606 may be configured to adapt the processors 1602 to perform at least a portion or a totality of the method 1100 of FIG. 11 and/or the method 1200 of FIG. 12. As another example, the machine-executable code 1606 may be configured to adapt the processors 1602 to perform at least a portion or a totality of the operations discussed for the computing device 110 of FIG. 1.

The processors 1602 may include a general purpose processor, a special purpose processor, a central processing unit (CPU), a microcontroller, a programmable logic controller (PLC), a digital signal processor (DSP), an application specific integrated circuit (ASIC), a field-programmable gate array (FPGA) or other programmable logic device, discrete gate or transistor logic, discrete hardware components, other programmable device, or any combination thereof designed to perform the functions disclosed herein. A general-purpose computer including a processor is considered a special-purpose computer while the general-purpose computer is configured to execute functional elements corresponding to the machine-executable code 1606 (e.g., software code, firmware code, hardware descriptions) related to embodiments of the present disclosure. It is noted that a general-purpose processor (may also be referred to herein as a host processor or simply a host) may be a microprocessor, but in the alternative, the processors 1602 may include any conventional processor, controller, microcontroller, or state machine. The processors 1602 may also be implemented as a combination of computing devices, such as a combination of a DSP and a microprocessor, a plurality of microprocessors, one or more microprocessors in conjunction with a DSP core, or any other such configuration.

In some embodiments the storage 1604 includes volatile data storage (e.g., random-access memory (RAM)), non-volatile data storage (e.g., Flash memory, a hard disc drive, a solid-state drive, erasable programmable read-only memory (EPROM), etc.). In some embodiments the processors 1602 and the storage 1604 may be implemented into a single device (e.g., a semiconductor device product, a system on chip (SOC), etc.). In some embodiments the processors 1602 and the storage 1604 may be implemented into separate devices.

In some embodiments the machine-executable code 1606 may include computer-readable instructions (e.g., software code, firmware code). By way of non-limiting example, the computer-readable instructions may be stored by the storage 1604, accessed directly by the processors 1602, and executed by the processors 1602 using at least the logic circuitry 1608. Also, by way of non-limiting example, the computer-readable instructions may be stored on the storage 1604, transferred to a memory device (not shown) for execution, and executed by the processors 1602 using at least the logic circuitry 1608. Accordingly, in some embodiments the logic circuitry 1608 includes electrically configurable logic circuitry 1608.

In some embodiments the machine-executable code 1606 may describe hardware (e.g., circuitry) to be implemented in logic circuitry 1608 to perform the functional elements. This hardware may be described at any of a variety of levels of abstraction, from low-level transistor layouts to high-level description languages. At a high-level of abstraction, a hardware description language (HDL) such as an IEEE Standard hardware description language (HDL) may be used. By way of non-limiting examples, VERILOG^{TM}, SYSTEMVERILOG^{TM} or very large-scale integration (VLSI) hardware description language (VHDL^{TM}) may be used.

HDL descriptions may be converted into descriptions at any of numerous other levels of abstraction as desired. As a non-limiting example, a high-level description can be converted to a logic-level description such as a register-transfer language (RTL), a gate-level (GL) description, a layout-level description, or a mask-level description. As a non-limiting example, micro-operations to be performed by hardware logic circuits (e.g., gates, flip-flops, registers, without limitation) of the logic circuitry 1608 may be described in a RTL and then converted by a synthesis tool into a GL description, and the GL description may be converted by a placement and routing tool into a layout-level description that corresponds to a physical layout of an integrated circuit of a programmable logic device, discrete gate or transistor logic, discrete hardware components, or combinations thereof. Accordingly, in some embodiments the machine-executable code 1606 may include an HDL, an RTL, a GL description, a mask level description, other hardware description, or any combination thereof.

In embodiments where the machine-executable code 1606 includes a hardware description (at any level of abstraction), a system (not shown, but including the storage 1604) may be configured to implement the hardware description described by the machine-executable code 1606. By way of non-limiting example, the processors 1602 may include a programmable logic device (e.g., an FPGA or a PLC) and the logic circuitry 1608 may be electrically controlled to implement circuitry corresponding to the hardware description into the logic circuitry 1608. Also by way of non-limiting example, the logic circuitry 1608 may include hard-wired logic manufactured by a manufacturing system (not shown, but including the storage 1604) according to the hardware description of the machine-executable code 1606.

Regardless of whether the machine-executable code 1606 includes computer-readable instructions or a hardware description, the logic circuitry 1608 is adapted to perform the functional elements described by the machine-executable code 1606 when implementing the functional elements of the machine-executable code 1606. It is noted that although a hardware description may not directly describe functional elements, a hardware description indirectly describes functional elements that the hardware elements described by the hardware description are capable of performing.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, etc.) of the computing system. In some embodiments, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated.

As used in the present disclosure, the term "combination" with reference to a plurality of elements may include a combination of all the elements or any of various different sub-combinations of some of the elements. For example, the phrase "A, B, C, D, or combinations thereof" may refer to any one of A, B, C, or D; the combination of each of A, B, C, and D; and any sub-combination of A, B, C, or D such as A, B, and C; A, B, and D; A, C, and D; B, C, and D; A and B; A and C; A and D; B and C; B and D; or C and D.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases "at least one" and "one or more" to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of "two recitations," without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

While the present disclosure has been described herein with respect to certain illustrated embodiments, those of ordinary skill in the art will recognize and appreciate that the present invention is not so limited. Rather, many additions, deletions, and modifications to the illustrated and described embodiments may be made without departing from the scope of the invention as hereinafter claimed along with their legal equivalents. In addition, features from one embodiment may be combined with features of another embodiment while still being encompassed within the scope of the invention as contemplated by the inventor.

What is claimed is:

1. A multi-laser spectroscopy system comprising:
a laser device including:
multiple laser sources configured to excite a laser light; and
an optical combiner disposed adjacent to the multiple laser sources and configured to combine the excited laser light from the multiple laser sources and output incident light;
a set of laser switches electrically connected to any of the multiple laser sources;
a cryochamber disposed adjacent to the laser device and configured to receive a sample and the incident light output from the optical combiner, wherein the incident light is configured to interact with the sample, thereby producing responsive light;
a spectrometer disposed adjacent to the cryochamber and configured to receive the responsive light and generate one or more measurement signals corresponding to the responsive light; and
a computing device electrically connected to the spectrometer and the set of laser switches, the computing device configured to cause the set of laser switches to individually control the excitation of the laser light from any of the multiple laser sources and to receive the one or more measurement signals from the spectrometer to determine one or more characteristics of the sample.

2. The multi-laser spectroscopy system of claim 1, wherein the computing device is further configured to initiate a sequence that causes the set of laser switches to individually control the excitation of the laser light from any of the multiple laser sources and thereby alter the incident light provided to the sample over a time period, and wherein the one or more measurement signals received at the spectrometer are configured to change during the time period.

3. The multi-laser spectroscopy system of claim 1, further comprising:
a bifurcated fiber including a probe tip to deliver the incident light to the cryochamber and receive the responsive light from the sample in response to the incident light to provide the responsive light to the spectrometer.

4. The multi-laser spectroscopy system of claim 1, wherein the multiple laser sources include a first laser source to produce first laser light having a center frequency corresponding to a wavelength of around 457 nm, a second laser source to produce second laser light having a center frequency corresponding to a wavelength of around 515 nm, a third laser source to produce third laser light having a center frequency corresponding to a wavelength of around 633 nm, and a fourth laser source to produce fourth laser light having a center frequency corresponding to a wavelength of around 830 nm.

5. The multi-laser spectroscopy system of claim 1, wherein the multiple laser sources include at least four laser sources.

6. The multi-laser spectroscopy system of claim 1, wherein the optical combiner includes a network of dichroic beam splitters.

7. The multi-laser spectroscopy system of claim 3, wherein the cryochamber includes:
a cryochamber housing defining the cryochamber, the probe tip extending through the cryochamber housing;
rails extending from the cryochamber housing;
a bracket including slots to receive the rails and a holder passage aligned with the probe tip responsive to the rails being received into the slots; and
a sample holder extending from the holder passage of the bracket toward the probe tip, the sample holder to secure the sample at its tip, the sample holder to place the sample in contact with the probe tip.

8. The multi-laser spectroscopy system of claim 7, wherein the bracket is shaped to define space between the bracket and the cryochamber with the sample deployed in contact with the probe tip.

9. The multi-laser spectroscopy system of claim 7, wherein the sample holder includes holder arms extending from a tip of the sample holder, the holder arms to secure the sample to the tip of the sample holder.

10. The multi-laser spectroscopy system of claim 7, wherein the sample holder includes an adhesive at a tip of the sample holder, the adhesive to secure the sample to the tip of the sample holder.

11. The multi-laser spectroscopy system of claim 7, wherein the sample includes a gemstone and the sample holder secures the gemstone with a table of the gemstone facing outward away from the sample holder.

12. The multi-laser spectroscopy system of claim 3, wherein the probe tip includes a tip of an excitation fiber surrounded by tips of detection fibers arranged in an annular pattern around the tip of the excitation fiber, the excitation fiber to extend from the optical combiner to the cryochamber, the detection fibers to extend from the cryochamber to the spectrometer.

13. The multi-laser spectroscopy system of claim 12, wherein tips of the detection fibers distal to the probe tip and proximal to the spectrometer are arranged in a linear array at an end of a spectrometer leg of the bifurcated fiber.

14. The multi-laser spectroscopy system of claim 11, wherein the one or more characteristics of the sample determined by the computing device include any of: an indication of whether the sample is natural or synthetic, or an indication of whether the sample is treated or untreated.

15. A method comprising:
providing a laser device including:
multiple laser sources configured to excite a laser light; and
an optical combiner disposed adjacent to the multiple laser sources and configured to combine the excited laser light from the multiple laser sources and output incident light;
disposing a cryochamber adjacent to the laser device, wherein the cryochamber is configured to receive a sample and the incident light output from the optical combiner, wherein the incident light is configured to interact with the sample, thereby producing responsive light;
connecting the cryochamber and a spectrometer via a bifurcated fiber including a probe tip to deliver the incident light to the cryochamber and receive the responsive light from the sample in response to the incident light to provide the responsive light to the spectrometer, the spectrometer configured to generate one or more measurement signals; and
electrically connecting a computing device to the spectrometer and a set of laser switches connected to any of the multiple laser sources, the computing device configured to cause the set of laser switches to individually control the excitation of the laser light from any of the multiple laser sources and to receive the one or more measurement signals from the spectrometer to determine one or more characteristics of the sample.

16. The method of claim 15, wherein the computing device is further configured to initiate a sequence that causes the set of laser switches to individually control the excitation of the laser light from any of the multiple laser sources and thereby alter the incident light provided to the sample over a time period, and wherein the one or more measurement signals received at the spectrometer are configured to change during the time period.

17. The method of claim 15, wherein the one or more characteristics of the sample determined by the computing device include any of: an indication of whether the sample is natural or synthetic, or an indication of whether the sample is treated or untreated.

18. A non-transitory, computer-readable storage medium having stored thereon a plurality of instructions, which, when executed by a processor of a computing device, cause the computing device to:
instruct a set of laser switches to individually control laser light excitation from any of multiple laser sources that are part of a laser device, wherein the laser device includes the multiple laser sources and an optical combiner configured to combine the excited laser light from the multiple laser sources and output incident light, wherein the incident light is directed to a sample in a cryochamber, thereby producing responsive light;
receive one or more measurement signals from a spectrometer configured to generate the one or more measurement signals from the responsive light; and
determine one or more characteristics of the sample based on the one or more measurement signals.

19. The computer-readable storage medium of claim 18, further causing the computing device to:
instruct the set of laser switches to control laser light excitation from different combinations of the multiple laser sources according to a sequence over a time period, wherein the one or more measurement signals received at the spectrometer are configured to change during the time period.

20. The computer-readable storage medium of claim 18, wherein the one or more characteristics of the sample determined by the computing device include any of: an indication of whether the sample is natural or synthetic, or an indication of whether the sample is treated or untreated.
